(19) United States
(12) Patent Application Publication
(10) Pub. No.: US 2025/0305035 A1
(43) Pub. Date: Oct. 2, 2025

(54) SINGLE EXTRACELLULAR VESICLE PROTEIN AND RNA ASSAY VIA IN-SITU FLUORESCENCE MICROSCOPY IN A UV MICROPATTERN ARRAY

(71) Applicant: OHIO STATE INNOVATION FOUNDATION, Columbus, OH (US)

(72) Inventors: Eduardo Reategui, Dublin, OH (US); Jingjing Zhang, Columbus, OH (US)

(21) Appl. No.: 18/717,941

(22) PCT Filed: Dec. 16, 2022
(86) PCT No.: PCT/US2022/081751

§ 371 Date: Jun. 7, 2024

Related U.S. Application Data
(60) Provisional application No. 63/290,386, filed on Dec. 16, 2021.

(51) Int. Cl. C12Q 1/6841; C12Q 1/6804; G01N 33/68

(52) U.S. Cl. CPC C12Q 1/6841; C12Q 1/6804; G01N 33/6803

(57) ABSTRACT

Described herein are systems and methods for determining the molecular cargo of an extracellular vesicle.

### CROSS-REFERENCE TO RELATED APPLICATIONS

This application is a U.S. national stage entry of International Patent Application No. PCT/US2022/081751, filed on Dec. 16, 2022, which claims priority to U.S. Provisional Patent Application No. 63/290,386 filed on Dec. 16, 2021, the entire contents of each of which are fully incorporated herein by reference.

### FEDERALLY SPONSORED RESEARCH

This invention was made with government support under grant number TR002884 awarded by the National Institutes of Health. The government has certain rights in the invention.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “029784-9120-US02_sequence_listing_xml_4 Dec. 2024,” was created on Dec. 4, 2024, contains 13 sequences, has a file size of 16.0 kilobytes (16,384 bytes), and is incorporated by reference in its entirety into the specification.

### BACKGROUND

Extracellular vesicles (EVs) are small membranous vesicles released by cells and are present in bodily fluids. EVs have been shown to play a role in different biological processes that span from physiological tissue regulation to pathogenic injury and organ remodeling. Despite the potential use of EVs in the clinic as diagnostic and therapeutic tools for different diseases, current methods for isolating and characterizing EVs are technically challenging. Isolation methods are usually cumbersome and irreproducible, while characterization relies on techniques including western blotting (WB), enzyme-linked immunosorbent assay (ELISA), polymerase chain reaction (PCR), next-generation sequencing (NGS), and mass spectroscopy (MS) which provide an average measurement of the nucleic acid and protein content. Consequently, with these characterization techniques, EVs are physically broken down to obtain their internal contents, whereby essential molecular information of tissue-specific single EVs (siEVs) can be lost. EVs are highly heterogeneous in molecular composition, with their proteins, RNAs, DNAs, lipids, and metabolites reflecting their tissue of origin. Investigating the molecular information within siEVs is necessary to understand the effects of EV-membrane proteins and vesicular cargo on EV-mediated intercellular signaling in diseases such as cancer. EVs have been shown to promote drug resistance, immunosuppression, the epithelial-to-mesenchymal transition (EMT), and metastasis. Therefore, there is a critical need to develop technologies that provide an accurate and efficient analysis of the molecular content within siEVs.

Several analytical methods have been reported to quantify the physical and molecular characteristics of siEVs. Nanoparticle tracking analysis (NTA) and tunable resistive pulse sensing (TRPS) are routinely used to measure the size and concentration of siEVs, with the minimum detectable size of EVs in the 70-100 nm range. However, NTA and TRPS lack specificity to characterize tissue-specific siEVs. Flow cytometry can detect siEVs as small as 40 nm, incorporating fluorescent protein detection. However, reduced multiplexed capability, aggregation or swarming of EVs due to the required concentrations, and extensive calibration requirements have limited their use. On the other hand, surface and cargo proteins have been characterized in siEVs using nano-plasmonic and interferometric biosensors. Moreover, antibody-DNA conjugates incorporating a random tag sequence in a proximity barcoding assay with NGS have been used to profile different proteins simultaneously in siEVs. Although these promising technologies have demonstrated their ability to resolve subpopulation of siEVs from different tissues, the complex cargo of EVs, such as nucleic acids, still requires strategies that enable different types of molecular cargo quantification.

Recently, super-resolution microscopy methods have been used to detect and quantify single proteins and nucleic acids at the sub-vesicular level to unravel the heterogeneity of EVs derived from biofluids. Quantitative single-molecule localization microscopy (qSMLM) can characterize the size and membrane protein content of siEVs from plasma. Stochastic optical reconstruction microscopy (STORM) combined with total internal fluorescence microscopy (TIRFM) has improved the signal-to-noise ratio and reduced the imaging time of siEVs. However, the nucleic acid cargo analysis of siEVs involves intricate chemistries that usually alter the native structure of EVs, producing high background signal levels, thus limiting the use of super-resolution microscopy to analyze highly expressed RNA biomarkers in siEVs. Moreover, the low-throughput nature of these techniques has also limited their broad dissemination for clinical use.

What is needed are systems and methods for determining the molecular cargo of an extracellular vesicle.

### SUMMARY

One embodiment described herein is a method of detecting a target type of molecular cargo with single extracellular vesicle resolution, the method comprising: coating a glass substrate with polyethylene glycol (PEG) to create a PEG-coated glass substrate; illuminating the PEG-coated glass substrate with UV light to create a micropattern array on the PEG-coated glass substrate; attaching one or more capture antibodies to the micropattern array; tethering a plurality of extracellular vesicles to the micropattern array via the one or more attached capture antibodies; binding detection antibodies and molecular beacons to the tethered extracellular vesicles on the micropattern array, wherein the detection antibodies are configured to bind to a first target type of molecular cargo (e.g., one or more proteins) and the molecular beacons are configured to bind to a second target type of molecular cargo (e.g., one or more RNAs); fluorescently imaging the micropattern array to capture image data; and detecting occurrences of individual extracellular vesicles expressing the first target type of molecular cargo based on fluorescent spots of a first color associated with the detection antibodies in the captured image data; detecting occurrences of individual extracellular vesicles expressing the second target type of molecular cargo based on fluorescent spots of a second color associated with the molecular beacons in the captured image data; and detecting occurrences of individual extracellular vesicles expressing both the first target type of molecular cargo and the second target type of molecular cargo based on fluorescent spots of a third color in the captured image data. In one aspect, the glass substrate is coated with poly-L-lysine (PLL) through physisorption prior to coating the glass substrate with PEG, wherein the PEG is covalently bound to the PLL through N-hydroxysuccinimide (NHS) chemistry. In another aspect, the PEG is methoxy-poly(ethylene glycol)-succinimidyl valerate (mPEG-SVA). In another aspect, the micropattern array comprises an array of circles, wherein each individual circle has a diameter ranging from about 10 μm to about 100 μm. In another aspect, each individual circle has a diameter of about 20 μm. In another aspect, the micropattern array comprises an array of circles, wherein each individual circle has a center-to-center spacing of about 80 μm in relation to an adjacent circle. In another aspect, the one or more capture antibodies are biotinylated and attach to the micropattern array by binding to a physisorbed avidin layer on the micropattern array. In another aspect, the first color associated with the detection antibodies in the captured image data is in a first channel (e.g., green), wherein the second color associated with the molecular beacons in the captured image data is in a second channel (e.g., red), and wherein the third color in the captured image data is in a third channel (e.g., yellow). In another aspect, the first target type of molecular cargo includes a target type of protein, wherein the detection antibodies include detection antibodies configured to bind the target type of protein, and wherein the second target type of molecular cargo includes a target type of RNA, wherein the molecular beacons include molecular beacons configured to bind the target type of RNA. In another aspect, the target type of RNA is selected from microRNA (miRNA), messenger RNA (mRNA), other RNA types, or combinations thereof. In another aspect, the detection antibodies are conjugated with one or more fluorophores for fluorescent imaging. In another aspect, the molecular beacons comprise one or more fluorescent dye sequences for fluorescent imaging. In another aspect, the molecular beacons comprise one or more locked nucleic acid (LNA) nucleotides to improve thermal stability and nuclease resistance. In another aspect, the molecular beacons are selected from any one of SEQ ID NO: 1-13. In another aspect, fluorescently imaging the micropattern array to capture image data comprises total internal reflection fluorescence microscopy (TIRFM). In another aspect, the plurality of extracellular vesicles is present at a concentration of about 1.0×10⁶ extracellular vesicles/mL or greater.

Another embodiment described herein is a method of detecting a target type of molecular cargo with single extracellular vesicle resolution, the method comprising: tethering a plurality of extracellular vesicles to a microarray; binding a first plurality of detection probes and a second plurality of detection probes to the plurality of extracellular vesicles on the microarray, wherein the first plurality of detection probes are configured to bind to a first target type of molecular cargo (e.g., one or more proteins) and to appear as a first color in fluorescent imaging (e.g., green), and wherein the second plurality of detection probes are configured to bind to a second target type of molecular cargo (e.g., one or more RNAs) and to appear as a second color in fluorescent imaging (e.g., red); using fluorescent imaging to capture image data of the extracellular vesicles in the microarray at a resolution sufficient to differentiate individual extracellular vesicles; detecting an occurrence of an extracellular vesicle expressing the first target type of molecular cargo by detecting a first color fluorescent spot (e.g., green) in the captured image data; detecting an occurrence of an extracellular vesicle expressing the second target type of molecular cargo by detecting a second color fluorescent spot (e.g., red) in the captured image data; and detecting an occurrence of an extracellular vesicle expressing both the first target type of molecular cargo and the second target type of molecular cargo by detecting a third color fluorescent spot (e.g., yellow) in the captured image data.

Another embodiment described herein is a system for detecting a target type of molecular cargo with single extracellular vesicle resolution, the system comprising: a polyethylene glycol (PEG)-coated glass substrate comprising a micropattern array having one or more capture antibodies attached thereto, wherein the one or more capture antibodies are configured to tether a plurality of extracellular vesicles; a plurality of detection antibodies and a plurality of molecular beacons, wherein the plurality of detection antibodies and the plurality of molecular beacons are configured to bind the plurality of extracellular vesicles, wherein the plurality of detection antibodies are configured to bind to a first target type of molecular cargo (e.g., one or more proteins), and wherein the plurality of molecular beacons are configured to bind to a second target type of molecular cargo (e.g., one or more RNAs); and a fluorescent imaging device to capture image data; wherein: occurrences of individual extracellular vesicles expressing the first target type of molecular cargo are detected based on fluorescent spots of a first color associated with the detection antibodies in the captured image data; occurrences of individual extracellular vesicles expressing the second target type of molecular cargo are detected based on fluorescent spots of a second color associated with the molecular beacons in the captured image data; and occurrences of individual extracellular vesicles expressing both the first target type of molecular cargo and the second target type of molecular cargo are detected based on fluorescent spots of a third color in the captured image data. In one aspect, the PEG is methoxy-poly(ethylene glycol)-succinimidyl valerate (mPEG-SVA). In another aspect, the micropattern array comprises an array of circles, wherein each individual circle has a diameter ranging from about 10 μm to about 100 μm. In another aspect, each individual circle has a diameter of about 20 μm. In another aspect, the micropattern array comprises an array of circles, wherein each individual circle has a center-to-center spacing of about 80 μm in relation to an adjacent circle. In another aspect, the one or more capture antibodies are biotinylated and attach to the micropattern array by binding to a physisorbed avidin layer on the micropattern array. In another aspect, the first color associated with the detection antibodies in the captured image data is in a first channel (e.g., green), wherein the second color associated with the molecular beacons in the captured image data is in a second channel (e.g., red), and wherein the third color in the captured image data is in a third channel (e.g., yellow). In another aspect, the first target type of molecular cargo includes a target type of protein, wherein the detection antibodies include detection antibodies configured to bind the target type of protein, and wherein the second target type of molecular cargo includes a target type of RNA, wherein the molecular beacons include molecular beacons configured to bind the target type of RNA. In another aspect, the target type of RNA is selected from microRNA (miRNA), messenger RNA (mRNA), other RNA types, or combinations thereof. In another aspect, the detection antibodies are conjugated with one or more fluorophores for fluorescent imaging. In another aspect, the molecular beacons comprise one or more fluorescent dye sequences for fluorescent imaging. In another aspect, the molecular beacons comprise one or more locked nucleic acid (LNA) nucleotides to improve thermal stability and nuclease resistance. In another aspect, the molecular beacons are selected from any one of SEQ ID NO: 1-13. In another aspect, the fluorescent imaging device is capable of performing total internal reflection fluorescence microscopy (TIRFM).

One embodiment described herein is a method of detecting different types of molecular cargo with single extracellular vesicle resolution (e.g., proteins or nucleic acids). A glass coverslip is coated with PEG, and UV light is used to create a micropattern array in the coated coverslip. Capture antibodies are used to tether a plurality of extracellular vesicles to the micropattern array and detection antibodies, and molecular beacons are applied to the extracellular vesicles. The detection antibodies are configured to bind to a first target type of molecular cargo (e.g., antigens or proteins), and the molecular beacons are configured to bind to a second target type of molecular cargo (e.g., microRNAs, messenger RNAs (mRNAs), or RNAs). The molecular cargo may include types of proteins and/or RNAs. Fluorescent imaging is then applied to captured image data of the micropattern array and occurrences of individual extracellular vesicles expressing the first target type of molecular cargo, the second target type of molecular cargo, or both are detected and quantified based on occurrences of fluorescent spots of one of three different colors in the captured image data. A first color associated with the detection antibodies indicates EVs expressing the first target type of molecular cargo, a second color associated with the molecular beacons indicates EVs expressing the second target type of molecular cargo, colocalization of both colors and visualized as a third different color indicates EVs simultaneously expressing both the first and second target types of molecular cargo.

In one embodiment, the two different detection probes (e.g., the detection antibodies and the molecular beacons) are configured to appear green and red, respectively, in fluorescent imaging and EVs that simultaneously express both the first and second target types of molecular cargo will appear as yellow spots in the fluorescent image data.

In another embodiment, the systems and methods described herein provide an in-situ fluorescent approach to detecting occurrences of EVs expressing one or more of multiple different molecular cargos (e.g., one or more proteins and/or RNA) with single-vesicle resolution. In various implementations, the type of molecular cargo that can be detected and analyzed is not constrained and both proteins and RNA can be detected and analyzed simultaneously. Additionally, “multiplexing” analysis can be used to identify single EVs that express both target types of molecular cargo. In some implementations, the systems and methods described herein are configured to simultaneously analyze a target type of protein and a target type of RNA, two different target types of protein, two different target types of RNA, or different portions of the same RNA.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 shows a flowchart of a method for detecting multiple types of molecular cargo of extracellular vesicles using a microarray according to one implementation.

FIG. 2 shows a block diagram of an example of an automated or semiautomated system configured to perform the method of FIG. 1.

FIG. 3A-H show single-EV detection with the siEVPRA integrated assay. FIG. 3A shows a schematic representation of the device fabrication with the PRIMO optical module to photoetch microdomains via digital micro device (DMD)-based UV projections, where NeutraAvidin (NA) was physisorbed to tether biotinylated antibodies against epitopes on the surfaces of siEV. FIG. 3B shows a schematic diagram of an example of an extracellular vesicle interacting with detection antibodies and molecular beacons in the method of FIG. 1. FIG. 3C shows a schematic diagram of an example of multiple extracellular vesicles interacting with the detection antibodies and the molecular beacons in a micropattern array according to the method of FIG. 1. FIG. 3D shows that CD63 (green dots) and miR-21 (red dots) on Gli36-derived siEVs are detected and colocalized (yellow dots) with the siEVPRA assay. The control sample (no EVs) demonstrates a negligible fluorescent signal. FIG. 3E shows that images are quantified as statistical distributions to depict the expression of CD63 and miR-21 at a single-vesicle level for the different samples. FIG. 3F shows quantification of relative fluorescence intensity (RFI) for CD63 and miR-21 in siEVs captured in the device. The control sample demonstrates different counts (n=3, error bars indicate the standard deviation). FIG. 3G shows scanning electron microscopy (SEM) of a typical sample confirming the presence of siEVs tethered to the surface of the device. FIG. 3H shows Gli36-derived siEV size distribution and concentration measured by TRPS demonstrating heterogeneity in particle size.

FIG. 4 shows homogenous NA physisorption. UV degradation of the PEG monolayer allows the homogenous adsorption of NA in distinct microdomains.

FIG. 5 shows EV recovery rate in different buffers. The concentration of EVs before and after incubation with a TE buffer and PBS at 4° C. and 37° C. demonstrates equal recovery rates (n=3, error bars indicate the standard deviation).

FIG. 6A-B show specificity of RNA detection via MBs. FIG. 6A shows miR-21 and miR-39 detected with the siEVPRA in Gli36-derived siEVs with and without partial permeabilization of the lipid membrane. FIG. 6B shows qualitative images quantified as RFIs on the Gli36-derived siEVs (n=3, error bars indicate the standard deviation).

FIG. 7A-B show cross-reactivity of engineered EVs. FIG. 7A shows Gli36-derived EVs transfected with cel-miR-39, cel-miR-54, and cel-miR-238 with the CNP biochip. FIG. 7B shows that the unmatched MB controls and the healthy serum control produced a low fluorescent signal.

FIG. 8A-E show specificity and sensitivity of RNA and protein detection. FIG. 8A shows that siEVs loaded with miR-54, miR-39, and miR-238 are detected by the corresponding MBs targeting miR-39 (green), miR-54 (red), and miR-238 (blue), whereas control samples (no EVs) demonstrated negligible fluorescent signal. FIG. 8B shows that the RFIs of EVs with miR-39, miR-54, and miR-238 with their corresponding MBs are higher than the different control conditions tested, including EVs with unmatched MBs (a1, a2, a3), EVs from human serum (a4), and no EVs (a5) (n=3, error bars indicate the standard deviation). FIG. 8C shows that the RFI for the detection of miR-39 in the engineered EVs increased with increasing concentrations of the cel-miR-39 plasmid transfected into the cells. EV concentrations were held constant at 1.0×10⁹ particles/mL for all conditions (n=3, error bars indicate the standard deviation). FIG. 8D shows the siEVPRA compared against a standard PCR test for detecting miR-39 from engineered EVs generated from transfected Gli36 cells with a plasmid concentration of 400 μg/uL (n=3, error bars indicate the standard deviation). Representative images and statistical distributions are provided in FIG. 9. FIG. 8E shows the ^{siEV}PRA compared against a standard ELISA for detecting EGFR from EVs isolated from Gli36 cells (n=3, error bars indicate the standard deviation). Representative images and statistical distributions are provided in FIG. 10.

FIG. 9A-B show sensitivity of the ^{siEV}PRA for RNA detection. FIG. 9A shows a serial dilution of engineered EVs enriched with miR-39 detected with the ^{siEV}PRA. FIG. 9B shows quantification of the qualitative images as statistical distributions.

FIG. 10A-B show sensitivity of the ^{siEV}PRA for protein detection. FIG. 10A shows a serial dilution of Gli36-derived siEVs enriched with EGFR detected with the ^{siEV}PRA. FIG. 10B shows quantification of the qualitative images as statistical distributions.

FIG. 11A-D show multiple target detection on AXL mRNA. FIG. 11A shows three regions of the AXL mRNA that were detected and multiplexed with the ^{siEV}PRA on Gli36-derived siEVs. FIG. 11B shows quantification of the qualitative images for the single region targets as statistical distributions. FIG. 11C shows quantification of the qualitative images for the single region targets as RFI (n=3, error bars indicate the standard deviation). FIG. 11D shows quantification of the qualitative images for all three targets as colocalization efficiencies for the A×L region combinations. C1, C2, and C3 represent the detected biomarkers from left to right (n=3, error bars indicate the standard deviation).

FIG. 12 shows cross-talk specificity of fluorescent channels. Fluorescently labeled antibodies, including CD63-488, CD81-640, and CD9-561 were illuminated by different wavelengths and excited only by their corresponding channel (n=3, error bars indicate the standard deviation).

FIG. 13A-C show simultaneous detection across various biomolecules. FIG. 13A shows EVs isolated from Gli36 cells tested with the ^{siEV}PRA for three different probes, including multiprotein detection (CD63, CD9, and CD81), mRNA-miRNA detection (AXL, miR-9-5p, and miR-21), and protein-miRNA detection (CD63, miR-9-5p, and miR-21). FIG. 13B shows quantification of colocalization efficiencies for the different biomarkers in Gli36-derived siEVs. C1, C2, and C3 represent the detected biomarkers from left to right (n=3, error bars indicate the standard deviation). FIG. 13C shows the RFIs of CD63, CD9, CD81, EGFR, GAPDH, miR-21, miR-9-5p, AXL, P53, and their corresponding controls, captured by CD63, CD9, EGFR, ARF6, Annexin A1, demonstrating a higher signal for the samples. EVs captured by IgG demonstrate similar signals among the biomarkers as the control (n=3, error bars indicate the standard deviation).

FIG. 14A-B show multiplexed RNA detection. FIG. 14A shows multiple miRNA, including miR-39 (green), miR-54 (red), and miR-238 (blue) multiplexed on siEVs with the ^{siEV}PRA. FIG. 14B shows quantification of the qualitative images as colocalization efficiencies for the miRNA in the Gli36-derived siEVs. C1, C2, and C3 represent the detected biomarkers from left to right (n=3, error bars indicate the standard deviation).

FIG. 15A-D show RNA sequencing of GBM-specific biomarkers and validation at a single-vesicle resolution. FIG. 15A-B show cellular and vesicular mRNA (FIG. 6A) and miRNA (FIG. 6B) sequenced across six GBM cell lines, including SF268, SF295, SF539, SNB19, SNB75, and U251, revealing the upregulation of NSF, miR-9-5p, NCAN, miR-1246-5p in cells and EVs. FIG. 15C shows NSF, miR-9-5p, NCAN, and miR-1246-5p profiled in EVs (solid line) and cells (dashed line) from the six different GBM cell lines by NGS bulk characterization (n=3). FIG. 15D shows NSF, miR-9-5p, NCAN, and miR-1246-5p profiled in siEVs from the six different GBM cell lines with the ^{siEV}PRA, showing upregulation of the four RNA biomarkers in comparison to the control samples (n=3, error bars indicate the standard deviation).

FIG. 16A-B show NSF detection with the ^{siEV}PRA. FIG. 16A shows qualitative images of the siEV detection for the expression of NSF on siEVs across the six GBM cell lines as detected by the ^{siEV}PRA. FIG. 16B shows statistical distributions for the expression of NSF on siEVs across the six GBM cell lines as detected by the ^{siEV}PRA.

FIG. 17A-B show NCAN detection with the ^{siEV}PRA. FIG. 17A shows qualitative images of the siEV detection for the expression of NCAN on siEVs across the six GBM cell lines as detected by the ^{siEV}PRA. FIG. 17B shows statistical distributions for the expression of NCAN on siEVs across the six GBM cell lines as detected by the ^{siEV}PRA.

FIG. 18A-B show miR-9-5p detection with the ^{siEV}PRA. FIG. 18A shows qualitative images of the siEV detection for the expression of miR-9-5p on siEVs across the six GBM cell lines as detected by the ^{siEV}PRA. FIG. 18B shows statistical distributions for the expression of miR-9-5p on siEVs across the six GBM cell lines as detected by the ^{siEV}PRA.

FIG. 19A-B show miR-1246-5p detection with the ^{siEV}PRA. FIG. 20A shows qualitative images of the siEV detection for the expression of miR-1246-5p on siEVs across the six GBM cell lines as detected by the ^{siEV}PRA. FIG. 19B shows statistical distributions for the expression of miR-1246-5p on siEVs across the six GBM cell lines as detected by the ^{siEV}PRA.

FIG. 20A-B show measurements of GBM-specific biomarkers at a single-vesicle resolution from GBM patient serum. FIG. 20A shows representative TIRFM images of siEV NSF, NCAN, miR-9-5p, and miR-1246-5p biomarkers from GBM patients (P) (n=10) and healthy donor (H) control serum (n=10) characterized with the ^{siEV}PRA. FIG. 20B shows that the RFI signals of NSF, miR-9-5p, NCAN, and miR-1246-5p in the GBM patient samples are higher than the RFI signals obtained for the healthy donor control samples (n=10).

FIG. 21 shows GBM serum-derived EV detection with the ^{siEV}PRA. Statistical distributions are shown for the siEV detection of NSF, miR-9-5p, miR-1246-5p, and NCAN for patient (P) and healthy donor (H) samples.

### DETAILED DESCRIPTION

Unless otherwise defined, all technical and scientific terms used herein have the same meaning as commonly understood by one of ordinary skill in the art. For example, any nomenclatures used in connection with, and techniques of biochemistry, molecular biology, immunology, microbiology, genetics, cell and tissue culture, and protein and nucleic acid chemistry described herein are well known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting of,” and “consisting essentially of,” the embodiments or elements presented herein, whether explicitly set forth or not.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to +10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to +10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the terms “molecular beacon” or “beacon” refer to single-stranded oligonucleotide hybridization probes that form a stem-and-loop “hairpin” structure. The loop contains a probe sequence that is complementary to a nucleic acid sequence, and the stem is formed by the annealing of complementary “arm” sequences that are located on either side of the probe sequence. The molecular beacon may comprise an internally quenched fluorophore whose fluorescence is restored when the molecular beacon binds to a target nucleic acid sequence. Exemplary molecular beacons contemplated for use in the systems and methods of the present invention are described in the Examples and presented in Table 2.

As used herein, the terms “target analyte,” “target biomarker,” “target type of molecular cargo,” “target type of protein,” or “target type of RNA” refer to a substance that is associated with a biological state or a biological process, such as a disease state or a diagnostic or prognostic indicator of a disease or disorder (e.g., an indicator identifying the likelihood of the existence or later development of a disease or disorder). The presence or absence of a biomarker, or the increase or decrease in the concentration of a biomarker, can be associated with and/or be indicative of a particular state or process. Biomarkers can include, but are not limited to, cells or cellular components (e.g., a viral cell, a bacterial cell, a fungal cell, a cancer cell, etc.), small molecules, lipids, vesicles, carbohydrates, nucleic acids, DNA, RNA, peptides, proteins, enzymes, antigens, and antibodies. A biomarker can be derived from an infectious agent, such as a bacterium, fungus or virus, or can be an endogenous molecule that is found in greater or lesser abundance in a subject suffering from a disease or disorder as compared to a healthy individual (e.g., an increase or decrease in expression of a gene or gene product).

As used herein, the terms “assay” or “bioassay” refer to a biochemical test for detecting the presence and/or measuring the concentration of a target analyte or target biomarker in a solution through the use of one or more biomolecules including, for example, capture and detection antibodies and/or molecular beacons.

As used herein, the term “configured to bind” generally refers to when an antibody or molecular beacon is adapted to bind to a target analyte or target biomarker more readily than it would bind to a random, unrelated biomolecule or analyte.

As used herein, the terms “microarray” or “micropattern array” refer to an array or a population of individual reaction sites that are formed and localized in spatially distinct and addressable locations on one or more substrate materials. In non-limiting exemplary embodiments of the present invention, a micropattern array may comprise an array of circular structures (i.e., circles) that are formed on a solid glass substrate and that are configured to bind to one or more target analytes or target biomarkers. For example, the micropattern array of circles may have one or more capture antibodies attached that are configured to bind and tether a plurality of extracellular vesicles.

Described herein is a single extracellular vesicle (EV) protein and RNA assay (siEVPRA) which is capable of multiplexing protein and RNA biomarker detection at a single-vesicle resolution. The assay consists of an array of microdomains patterned on a polyethylene glycol (PEG)-coated glass surface using UV light with a digital-micromirror device (DMD) that allows maskless photopatterning. The arrayed surface is functionalized with antibodies against EV-specific epitopes, such as tetraspanins, ADP-ribosylation factor 6 (ARF6), and Annexin A1 to immobilize subpopulations of siEVs onto distinct positions. Fluorescently labeled antibodies and RNA-targeting molecular beacons (MBs) are used to generate signals for proteins, mRNAs, and miRNAs on siEVs detected by TIRFM and quantified via automatic image acquisition. The siEVPRA exceeds the detection limit for both ELISA and PCR by three orders of magnitude without tedious EV lysis extraction procedures. The ability of the ^{siEV}PRA to multiplex various biomarkers within and across biomolecule species enables complex EV heterogeneity analyses such as simultaneous protein and RNA detection of up to 9 different biomarkers in siEVs (4 proteins and 5 RNAs) enriched with different capture antibodies. In this work, the ^{siEV}PRA was extended to investigate subpopulations of EVs from glioblastoma (GBM) cell lines to determine the heterogeneity of different RNAs, confirmed with bulk RNA sequencing. Next, the clinical utility of the ^{siEV}PRA was established by validating the expression of different mRNAs and miRNAs associated with GBM in siEVs. The siEV analysis of serum from GBM patients demonstrated that distinctive RNA signals were obtained when compared to healthy controls. This is believed to be the first assay that enables the simultaneous and low-dose profiling of protein, miRNA, and mRNA on siEVs, lending unique applications for liquid biopsies and therapeutics.

The physical and molecular heterogeneity of EVs confounds bulk biomarker characterization and encourages the development of novel assays capable of profiling EVs at a single-vesicle resolution. Some implementations described in the examples herein provide the siEVPRA to simultaneously detect proteins, messenger RNAs (mRNA), and microRNAs (miRNA) in sEVs. In some implementations, the SiEVPRA includes an array of microdomains on a polyethylene glycol (PEG)-coated glass surface produced via maskless UV photopatterning. The arrayed surface is functionalized with antibodies to target sEV subpopulations. Fluorescently-labeled antibodies and RNA-targeting molecular beacons (MBs) are used to generate signals for target proteins, mRNAs, and/or miRNAs on sEVs that are then detected by total internal reflection fluorescence microscopy (TIRFM). The siEVPRA can detect low-dose EVs in 20 μL of biofluid due to its high specificity and sensitivity, outperforming ELISA and PCR by five orders of magnitude.

In some implementations, the ^{siEV}PRA is used to analyze EVs harvested from glioblastoma multiforme (GBM) cell lines. In some implementations, the ^{siEV}PRA may be implemented in clinical tools configured to detect different mRNAs and miRNAs that are specifically associated with GBM, lung, and breast cancer. Thus, the ^{siEV}PRA may be used in various implementations to detect a disease (e.g., cancer), to monitor the progression of a disease and/or treatment of the disease, and/or for researching heterogeneity of proteins and RNAs in subpopulations of EVs.

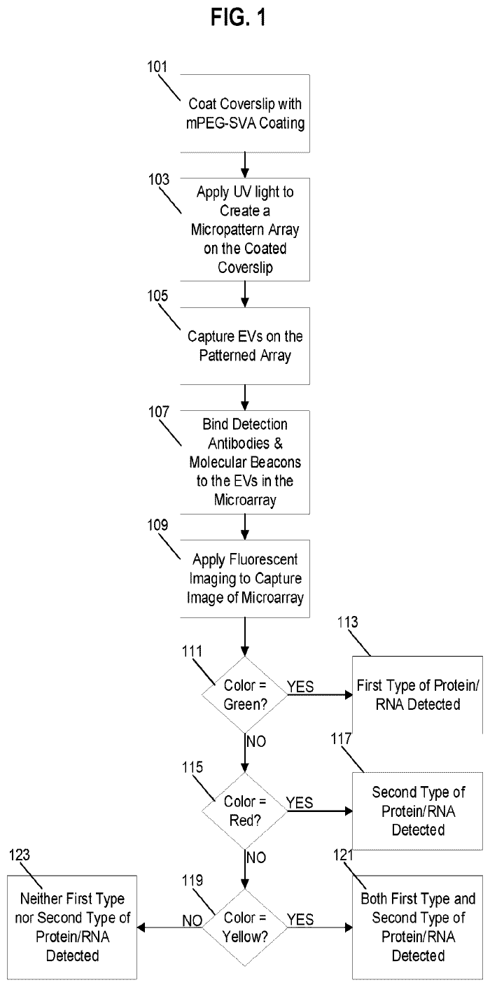

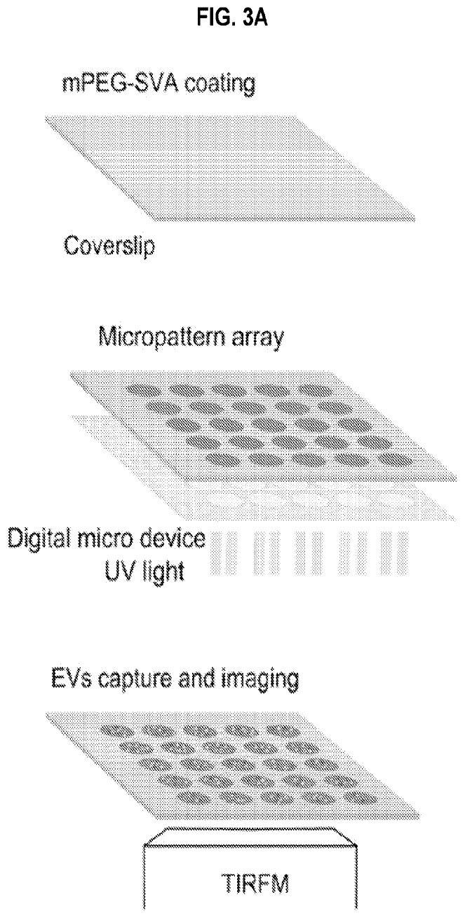

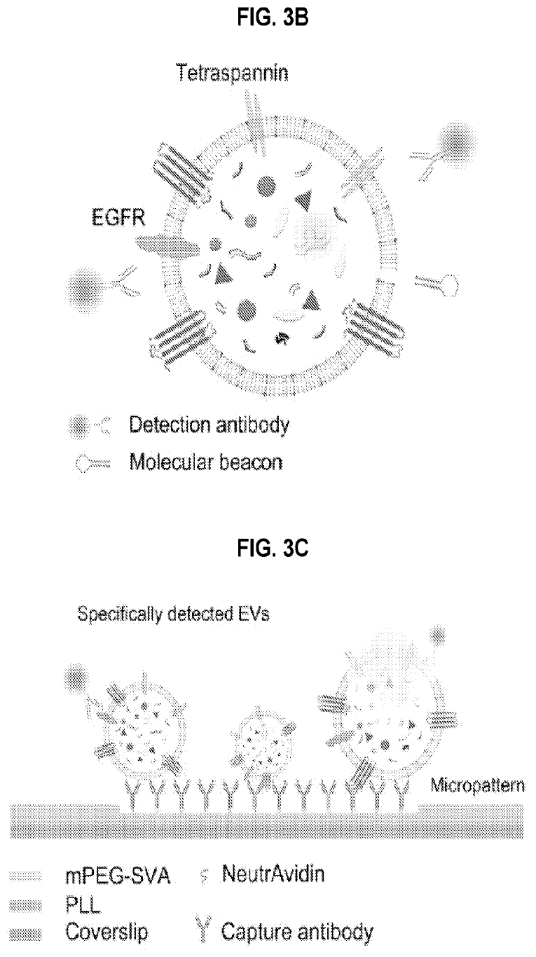

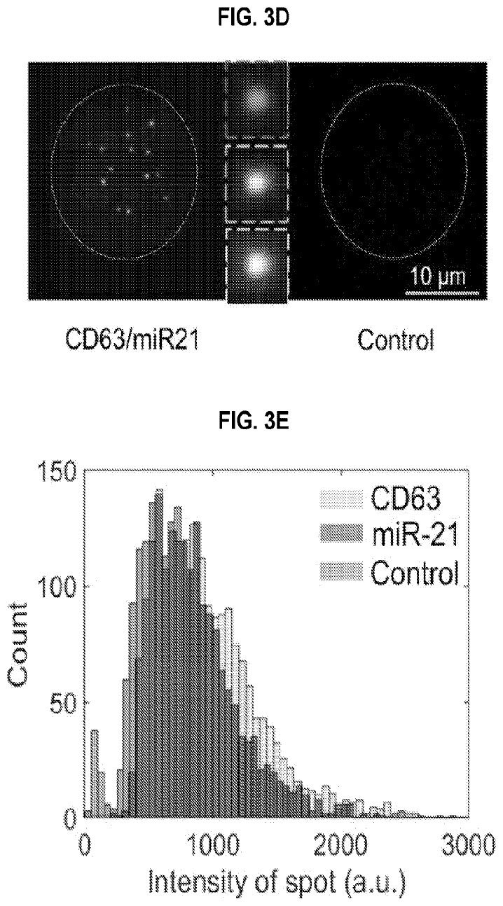

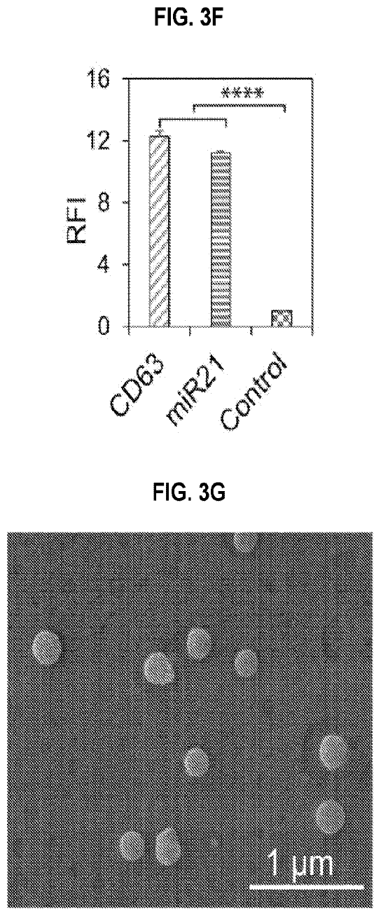

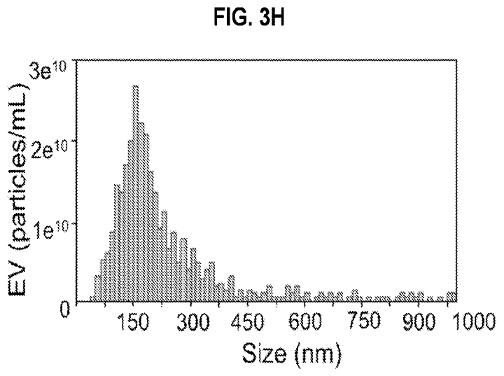

FIG. 1 illustrates an exemplary method for preparing and using the ^{siEV}PRA to detect occurrences of two different types of molecules (e.g., proteins and/or RNAs) in extracellular vesicles. A glass coverslip is coated with PLL through physisorption and mPEG-SVA is covalently attached to the surface through N-hydroxysuccinimide (NHS) chemistry (step 101). FIG. 3A illustrates an example of a coated glass coverslip. An array of circles (e.g., 20 μm diameter circles) is then cleaved from the mPEG non-biofouling monolayer via UV projections translated by a DMD in the presence of PLPP as a photoactivator (step 103). The middle panel of FIG. 3A illustrates one example of a process for micropatterning the coated coverslip. Biotinylated antibodies against CD63 and CD9, EGFR, ARF6, and AnnexA1, which are present as membrane proteins on EVs, are then patterned in the microdomains of the five-by-five array to selectively tether sEVs (step 105).

A fluorophore-conjugated antibody and a molecular beacon, each selected to detect a particular type of molecular cargo (e.g., a specific protein or RNA) are then applied to the tethered sEVs on the array and used as detection probes (step 107). For example, a fluorophore-conjugated antibody against CD63 and a molecular beacon targeting miR-21 (an abundant EV-enveloped miRNA) may be selected as the detection probes. The microarray is then visualized using total internal reflection fluorescence microscopy (TIRFM) to capture fluorescence image data of the microarray including the tethered sEVs and the selectively bound detection probes (step 109) as illustrated in the bottom panel of FIG. 3A. FIG. 3B illustrates an example of a single EV interacting with fluorescent detection antibodies and molecular beacons and FIG. 3C illustrates an example of multiple individual EVs tethered to an array.

The fluorescent color associated with the selected detection probes is then used to determine the presence or absence of the target molecular cargo. For example, the detection probes may be selected such that instances of a first detection probe (e.g., the fluorophore-conjugated antibody) will appear green in fluorescence imaging and instances of the second detection probe (e.g., the molecular beacon) will appear red in fluorescence imaging. This fluorescence imaging approach also provides a “multiplexing” mechanism as the simultaneous presence of both green fluorescent light and red fluorescent light will appear as yellow fluorescent light in the image data. Accordingly, a green fluorescent spot in the captured image data (step 111) indicates a single EV expressing the molecular cargo associated with the first detection probe (e.g., CD63) (step 113) and not expressing the molecular cargo associated with the second detection probe (e.g., miR-21). Similarly, a red fluorescent spot in the captured image data (step 115) indicates a single EV carrying the molecular cargo associated with the second detection probe (e.g., miR-21) (step 117) and not the molecular cargo associated with the first detection probe (e.g., CD63). A yellow fluorescent spot in the captured image data (step 119) indicates colocalization of both biomarkers in the same single EV (step 121) (i.e., both the molecular cargo associated with the first detection probe and the molecular cargo associated with the second detection probe). Conversely, the absence of red, green, or yellow fluorescent spots (or fluorescent signals below a defined intensity level) indicates that neither the molecular cargo associated with the first detection probe, nor the molecular cargo associated with the second detection probe, are present in a particular EV (step 123).

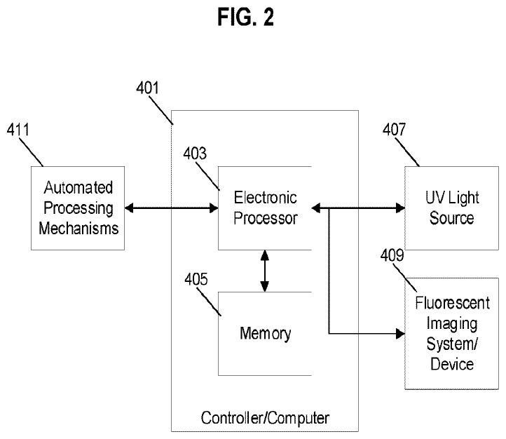

In some implementations, the mechanisms such as, for example, producing the microarray, tethering EVs to the microarray, and/or capturing/analyzing the image data may be performed manually, may be fully automated, or may be semi-automated. FIG. 2 illustrates an example of a system configured to perform the method of FIG. 1 in an automated or semi-automated manner. The system includes an electronic controller or computer 401 that includes an electronic processor 403 and a non-transitory computer-readable memory 405. The memory 405 stores data and instructions that, when executed by the electronic processor 403, provides the functionality of the controller/computer 401. The electronic processor 403 is communicative coupled to a UV light source 407 and configured to transmit control signals to the UV light source 407 which cause the UV light source to form the array pattern in the coated coverslip. The electronic processor 403 is also communicatively coupled to a fluorescent imaging system/device 409 (e.g., the TIRFM discussed in the examples above) and configured to cause the fluorescent imaging system/device 409 to capture fluorescent image data of the microarray and to then receive the captured image data from the fluorescent imaging system/device 409.

In some implementations, the electronic processor 403 may also be communicatively coupled to one or more additional automated processing mechanism 411 configured to perform other automated or semi-automated tasks associated with the method of FIG. 1. For example, the automated processing mechanisms may include a robotic mechanism and/or fluid handling mechanisms for moving the glass coverslip and/or dispensing the various agents (e.g., the mPEG-SVA, the capture antibodies, the EVs, and/or the detection probes).

Described herein are systems, and methods for simultaneously detecting the presence or absence of multiple different types of molecular cargos (e.g., one or more proteins and/or RNA) in single extracellular vesicles using a UV micropatterned array and in-situ fluorescence microscopy.

One embodiment described herein is a method of detecting a target type of molecular cargo with single extracellular vesicle resolution, the method comprising: coating a glass substrate with polyethylene glycol (PEG) to create a PEG-coated glass substrate; illuminating the PEG-coated glass substrate with UV light to create a micropattern array on the PEG-coated glass substrate; attaching one or more capture antibodies to the micropattern array; tethering a plurality of extracellular vesicles to the micropattern array via the one or more attached capture antibodies; binding detection antibodies and molecular beacons to the tethered extracellular vesicles on the micropattern array, wherein the detection antibodies are configured to bind to a first target type of molecular cargo (e.g., one or more proteins) and the molecular beacons are configured to bind to a second target type of molecular cargo (e.g., one or more RNAs); fluorescently imaging the micropattern array to capture image data; and detecting occurrences of individual extracellular vesicles expressing the first target type of molecular cargo based on fluorescent spots of a first color associated with the detection antibodies in the captured image data; detecting occurrences of individual extracellular vesicles expressing the second target type of molecular cargo based on fluorescent spots of a second color associated with the molecular beacons in the captured image data; and detecting occurrences of individual extracellular vesicles expressing both the first target type of molecular cargo and the second target type of molecular cargo based on fluorescent spots of a third color in the captured image data. In one aspect, the glass substrate is coated with poly-L-lysine (PLL) through physisorption prior to coating the glass substrate with PEG, wherein the PEG is covalently bound to the PLL through N-hydroxysuccinimide (NHS) chemistry. In another aspect, the PEG is methoxy-poly(ethylene glycol)-succinimidyl valerate (mPEG-SVA). In another aspect, the micropattern array comprises an array of circles, wherein each individual circle has a diameter ranging from about 5 μm to about 100 μm. In another aspect, each individual circle has a diameter ranging from about 10 μm to about 100 μm. In another aspect, each individual circle has a diameter ranging from about 10 μm to about 50 μm. In another aspect, each individual circle has a diameter ranging from about 15 μm to about 30 μm. In another aspect, each individual circle has a diameter ranging from about 18 μm to about 22 μm. In another aspect, each individual circle has a diameter of about 20 μm. In another aspect, the micropattern array comprises an array of circles, wherein each individual circle has a center-to-center spacing ranging from about 20 μm to about 150 μm in relation to an adjacent circle. In another aspect, each individual circle has a center-to-center spacing ranging from about 40 μm to about 120 μm in relation to an adjacent circle. In another aspect, each individual circle has a center-to-center spacing ranging from about 50 μm to about 100 μm in relation to an adjacent circle. In another aspect, each individual circle has a center-to-center spacing ranging from about 60 μm to about 90 μm in relation to an adjacent circle. In another aspect, each individual circle has a center-to-center spacing ranging from about 65 μm to about 85 μm in relation to an adjacent circle. In another aspect, each individual circle has a center-to-center spacing ranging from about 75 μm to about 85 μm in relation to an adjacent circle. In another aspect, each individual circle has a center-to-center spacing of about 80 μm in relation to an adjacent circle. In another aspect, the one or more capture antibodies are biotinylated and attach to the micropattern array by binding to a physisorbed avidin layer on the micropattern array. In another aspect, the first color associated with the detection antibodies in the captured image data is in a first channel (e.g., green), wherein the second color associated with the molecular beacons in the captured image data is in a second channel (e.g., red), and wherein the third color in the captured image data is in a third channel (e.g., yellow). In another aspect, the first target type of molecular cargo includes a target type of protein, wherein the detection antibodies include detection antibodies configured to bind the target type of protein, and wherein the second target type of molecular cargo includes a target type of RNA, wherein the molecular beacons include molecular beacons configured to bind the target type of RNA. In another aspect, the target type of RNA is selected from microRNA (miRNA), messenger RNA (mRNA), other RNA types, or combinations thereof. In another aspect, the detection antibodies are conjugated with one or more fluorophores for fluorescent imaging. In another aspect, the molecular beacons comprise one or more fluorescent dye sequences for fluorescent imaging. In another aspect, the molecular beacons comprise one or more locked nucleic acid (LNA) nucleotides to improve thermal stability and nuclease resistance. In another aspect, the molecular beacons have at least 90-99% identity to any one of SEQ ID NO: 1-13. In another aspect, the molecular beacons are selected from any one of SEQ ID NO: 1-13. In another aspect, fluorescently imaging the micropattern array to capture image data comprises total internal reflection fluorescence microscopy (TIRFM). In another aspect, the plurality of extracellular vesicles is present at a concentration of about 1.0×10⁵ extracellular vesicles/mL or greater. In another aspect, the plurality of extracellular vesicles is present at a concentration of about 1.0×10⁶ extracellular vesicles/mL or greater. In another aspect, the plurality of extracellular vesicles is present at a concentration of about 1.0×10⁷ extracellular vesicles/mL or greater.

Another embodiment described herein is a method of detecting a target type of molecular cargo with single extracellular vesicle resolution, the method comprising: tethering a plurality of extracellular vesicles to a microarray; binding a first plurality of detection probes and a second plurality of detection probes to the plurality of extracellular vesicles on the microarray, wherein the first plurality of detection probes are configured to bind to a first target type of molecular cargo (e.g., one or more proteins) and to appear as a first color in fluorescent imaging (e.g., green), and wherein the second plurality of detection probes are configured to bind to a second target type of molecular cargo (e.g., one or more RNAs) and to appear as a second color in fluorescent imaging (e.g., red); using fluorescent imaging to capture image data of the extracellular vesicles in the microarray at a resolution sufficient to differentiate individual extracellular vesicles; detecting an occurrence of an extracellular vesicle expressing the first target type of molecular cargo by detecting a first color fluorescent spot (e.g., green) in the captured image data; detecting an occurrence of an extracellular vesicle expressing the second target type of molecular cargo by detecting a second color fluorescent spot (e.g., red) in the captured image data; and detecting an occurrence of an extracellular vesicle expressing both the first target type of molecular cargo and the second target type of molecular cargo by detecting a third color fluorescent spot (e.g., yellow) in the captured image data.

Another embodiment described herein is a system for detecting a target type of molecular cargo with single extracellular vesicle resolution, the system comprising: a polyethylene glycol (PEG)-coated glass substrate comprising a micropattern array having one or more capture antibodies attached thereto, wherein the one or more capture antibodies are configured to tether a plurality of extracellular vesicles; a plurality of detection antibodies and a plurality of molecular beacons, wherein the plurality of detection antibodies and the plurality of molecular beacons are configured to bind the plurality of extracellular vesicles, wherein the plurality of detection antibodies are configured to bind to a first target type of molecular cargo (e.g., one or more proteins), and wherein the plurality of molecular beacons are configured to bind to a second target type of molecular cargo (e.g., one or more RNAs); and a fluorescent imaging device to capture image data; wherein: occurrences of individual extracellular vesicles expressing the first target type of molecular cargo are detected based on fluorescent spots of a first color associated with the detection antibodies in the captured image data; occurrences of individual extracellular vesicles expressing the second target type of molecular cargo are detected based on fluorescent spots of a second color associated with the molecular beacons in the captured image data; and occurrences of individual extracellular vesicles expressing both the first target type of molecular cargo and the second target type of molecular cargo are detected based on fluorescent spots of a third color in the captured image data. In one aspect, the PEG is methoxy-poly(ethylene glycol)-succinimidyl valerate (mPEG-SVA). In another aspect, the micropattern array comprises an array of circles, wherein each individual circle has a diameter ranging from about 5 μm to about 100 μm. In another aspect, each individual circle has a diameter ranging from about 10 μm to about 100 μm. In another aspect, each individual circle has a diameter ranging from about 10 μm to about 50 μm. In another aspect, each individual circle has a diameter ranging from about 15 μm to about 30 μm. In another aspect, each individual circle has a diameter ranging from about 18 μm to about 22 μm. In another aspect, each individual circle has a diameter of about 20 μm. In another aspect, the micropattern array comprises an array of circles, wherein each individual circle has a center-to-center spacing ranging from about 20 μm to about 150 μm in relation to an adjacent circle. In another aspect, each individual circle has a center-to-center spacing ranging from about 40 μm to about 120 μm in relation to an adjacent circle. In another aspect, each individual circle has a center-to-center spacing ranging from about 50 μm to about 100 μm in relation to an adjacent circle. In another aspect, each individual circle has a center-to-center spacing ranging from about 60 μm to about 90 μm in relation to an adjacent circle. In another aspect, each individual circle has a center-to-center spacing ranging from about 65 μm to about 85 μm in relation to an adjacent circle. In another aspect, each individual circle has a center-to-center spacing ranging from about 75 μm to about 85 μm in relation to an adjacent circle. In another aspect, each individual circle has a center-to-center spacing of about 80 μm in relation to an adjacent circle. In another aspect, the one or more capture antibodies are biotinylated and attach to the micropattern array by binding to a physisorbed avidin layer on the micropattern array. In another aspect, the first color associated with the detection antibodies in the captured image data is in a first channel (e.g., green), wherein the second color associated with the molecular beacons in the captured image data is in a second channel (e.g., red), and wherein the third color in the captured image data is in a third channel (e.g., yellow). In another aspect, the first target type of molecular cargo includes a target type of protein, wherein the detection antibodies include detection antibodies configured to bind the target type of protein, and wherein the second target type of molecular cargo includes a target type of RNA, wherein the molecular beacons include molecular beacons configured to bind the target type of RNA. In another aspect, the target type of RNA is selected from microRNA (miRNA), messenger RNA (mRNA), other RNA types, or combinations thereof. In another aspect, the detection antibodies are conjugated with one or more fluorophores for fluorescent imaging. In another aspect, the molecular beacons comprise one or more fluorescent dye sequences for fluorescent imaging. In another aspect, the molecular beacons comprise one or more locked nucleic acid (LNA) nucleotides to improve thermal stability and nuclease resistance. In another aspect, the molecular beacons have at least 90-99% identity to any one of SEQ ID NO: 1-13. In another aspect, the molecular beacons are selected from any one of SEQ ID NO: 1-13. In another aspect, the fluorescent imaging device is capable of performing total internal reflection fluorescence microscopy (TIRFM).

Further embodiments described herein include nucleic acid molecules comprising polynucleotides having nucleotide sequences about 50%, 55%, 60%, 65%, 70%, 75%, 80%, 85%, 90%, 91%, 92%, 93%, 94%, 95%, 96%, 97%, 98%, or 99% identical, and more preferably at least about 90-99% or 100% identical to (a) nucleotide sequences, or degenerate, homologous, or codon-optimized variants thereof, having the nucleotide sequences in SEQ ID NO: 1-13; or (b) nucleotide sequences capable of hybridizing to the complement of any of the nucleotide sequences in (a).

By a polynucleotide having a nucleotide sequence at least, for example, 90-99% “identical” to a reference nucleotide sequence intended that the nucleotide sequence of the polynucleotide be identical to the reference sequence except that the polynucleotide sequence can include up to about 10 to 1 point mutations, additions, or deletions per each 100 nucleotides of the reference nucleotide sequence.

In other words, to obtain a polynucleotide having a nucleotide sequence about at least 90-99% identical to a reference nucleotide sequence, up to 10% of the nucleotides in the reference sequence can be deleted, added, or substituted, with another nucleotide, or a number of nucleotides up to 10% of the total nucleotides in the reference sequence can be inserted into the reference sequence. These mutations of the reference sequence can occur at 5′- or 3′-terminal positions of the reference nucleotide sequence or anywhere between those terminal positions, interspersed either individually among nucleotides in the reference sequence or in one or more contiguous groups within the reference sequence. The same is applicable to polypeptide sequences about at least 90-99% identical to a reference polypeptide sequence.

As noted above, two or more polynucleotide sequences can be compared by determining their percent identity. Two or more amino acid sequences likewise can be compared by determining their percent identity. The percent identity of two sequences, whether nucleic acid or peptide sequences, is generally described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:4 82-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14 (6): 6745-6763 (1986).

Another embodiment described herein is a research tool comprising the nucleotide sequences described herein.

Another embodiment described herein is a reagent comprising the nucleotide sequences described herein.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:

Clause 1. A method of detecting a target type of molecular cargo with single extracellular vesicle resolution, the method comprising:
coating a glass substrate with polyethylene glycol (PEG) to create a PEG-coated glass substrate;
illuminating the PEG-coated glass substrate with UV light to create a micropattern array on the PEG-coated glass substrate;
attaching one or more capture antibodies to the micropattern array;
tethering a plurality of extracellular vesicles to the micropattern array via the one or more attached capture antibodies;
binding detection antibodies and molecular beacons to the tethered extracellular vesicles on the micropattern array, wherein the detection antibodies are configured to bind to a first target type of molecular cargo and the molecular beacons are configured to bind to a second target type of molecular cargo;
fluorescently imaging the micropattern array to capture image data; and
detecting occurrences of individual extracellular vesicles expressing the first target type of molecular cargo based on fluorescent spots of a first color associated with the detection antibodies in the captured image data;
detecting occurrences of individual extracellular vesicles expressing the second target type of molecular cargo based on fluorescent spots of a second color associated with the molecular beacons in the captured image data; and
detecting occurrences of individual extracellular vesicles expressing both the first target type of molecular cargo and the second target type of molecular cargo based on fluorescent spots of a third color in the captured image data.

Clause 2. The method of clause 1, wherein the glass substrate is coated with poly-L-lysine (PLL) through physisorption prior to coating the glass substrate with PEG, wherein the PEG is covalently bound to the PLL through N-hydroxysuccinimide (NHS) chemistry.

Clause 3. The method of clause 1 or 2, wherein the PEG is methoxy-poly(ethylene glycol)-succinimidyl valerate (mPEG-SVA).

Clause 4. The method of any one of clauses 1-3, wherein the micropattern array comprises an array of circles, wherein each individual circle has a diameter ranging from about 10 μm to about 100 μm.

Clause 5. The method of any one of clauses 1-4, wherein each individual circle has a diameter of about 20 μm.

Clause 6. The method of any one of clauses 1-5, wherein the micropattern array comprises an array of circles, wherein each individual circle has a center-to-center spacing of about 80 μm in relation to an adjacent circle.

Clause 7. The method of any one of clauses 1-6, wherein the one or more capture antibodies are biotinylated and attach to the micropattern array by binding to a physisorbed avidin layer on the micropattern array.

Clause 8. The method of any one of clauses 1-7, wherein the first color associated with the detection antibodies in the captured image data is in a first channel, wherein the second color associated with the molecular beacons in the captured image data is in a second channel, and wherein the third color in the captured image data is in a third channel.

Clause 9. The method of any one of clauses 1-8, wherein the first target type of molecular cargo includes a target type of protein, wherein the detection antibodies include detection antibodies configured to bind the target type of protein, and wherein the second target type of molecular cargo includes a target type of RNA, wherein the molecular beacons include molecular beacons configured to bind the target type of RNA.

Clause 10. The method of any one of clauses 1-9, wherein the target type of RNA is selected from microRNA (miRNA), messenger RNA (mRNA), other RNA types, or combinations thereof.

Clause 11. The method of any one of clauses 1-10, wherein the detection antibodies are conjugated with one or more fluorophores for fluorescent imaging.

Clause 12. The method of any one of clauses 1-11, wherein the molecular beacons comprise one or more fluorescent dye sequences for fluorescent imaging.

Clause 13. The method of any one of clauses 1-12, wherein the molecular beacons comprise one or more locked nucleic acid (LNA) nucleotides to improve thermal stability and nuclease resistance.

Clause 14. The method of any one of clauses 1-13, wherein the molecular beacons are selected from any one of SEQ ID NO: 1-13.

Clause 15. The method of any one of clauses 1-14, wherein fluorescently imaging the micropattern array to capture image data comprises total internal reflection fluorescence microscopy (TIRFM).

Clause 16. The method of any one of clauses 1-15, wherein the plurality of extracellular vesicles is present at a concentration of about 1.0×10⁶ extracellular vesicles/mL or greater.

Clause 17. A method of detecting a target type of molecular cargo with single extracellular vesicle resolution, the method comprising:
tethering a plurality of extracellular vesicles to a microarray;
binding a first plurality of detection probes and a second plurality of detection probes to the plurality of extracellular vesicles on the microarray, wherein the first plurality of detection probes are configured to bind to a first target type of molecular cargo and to appear as a first color in fluorescent imaging, and wherein the second plurality of detection probes are configured to bind to a second target type of molecular cargo and to appear as a second color in fluorescent imaging;
using fluorescent imaging to capture image data of the extracellular vesicles in the microarray at a resolution sufficient to differentiate individual extracellular vesicles;
detecting an occurrence of an extracellular vesicle expressing the first target type of molecular cargo by detecting a first color fluorescent spot in the captured image data;
detecting an occurrence of an extracellular vesicle expressing the second target type of molecular cargo by detecting a second color fluorescent spot in the captured image data; and
detecting an occurrence of an extracellular vesicle expressing both the first target type of molecular cargo and the second target type of molecular cargo by detecting a third color fluorescent spot in the captured image data.

Clause 18. A system for detecting a target type of molecular cargo with single extracellular vesicle resolution, the system comprising:
a polyethylene glycol (PEG)-coated glass substrate comprising a micropattern array having one or more capture antibodies attached thereto, wherein the one or more capture antibodies are configured to tether a plurality of extracellular vesicles;
a plurality of detection antibodies and a plurality of molecular beacons, wherein the plurality of detection antibodies and the plurality of molecular beacons are configured to bind the plurality of extracellular vesicles, wherein the plurality of detection antibodies are configured to bind to a first target type of molecular cargo, and wherein the plurality of molecular beacons are configured to bind to a second target type of molecular cargo; and
a fluorescent imaging device to capture image data;
wherein:
   occurrences of individual extracellular vesicles expressing the first target type of molecular cargo are detected based on fluorescent spots of a first color associated with the detection antibodies in the captured image data;
   occurrences of individual extracellular vesicles expressing the second target type of molecular cargo are detected based on fluorescent spots of a second color associated with the molecular beacons in the captured image data; and
   occurrences of individual extracellular vesicles expressing both the first target type of molecular cargo and the second target type of molecular cargo are detected based on fluorescent spots of a third color in the captured image data.

Clause 19. The system of clause 18, wherein the PEG is methoxy-poly(ethylene glycol)-succinimidyl valerate (mPEG-SVA).

Clause 20. The system of clause 18 or 19, wherein the micropattern array comprises an array of circles, wherein each individual circle has a diameter ranging from about 10 μm to about 100 μm.

Clause 21. The system of any one of clauses 18-20, wherein each individual circle has a diameter of about 20 μm.

Clause 22. The system of any one of clauses 18-21, wherein the micropattern array comprises an array of circles, wherein each individual circle has a center-to-center spacing of about 80 μm in relation to an adjacent circle.

Clause 23. The system of any one of clauses 18-22, wherein the one or more capture antibodies are biotinylated and attach to the micropattern array by binding to a physisorbed avidin layer on the micropattern array.

Clause 24. The system of any one of clauses 18-23, wherein the first color associated with the detection antibodies in the captured image data is in a first channel, wherein the second color associated with the molecular beacons in the captured image data is in a second channel, and wherein the third color in the captured image data is in a third channel.

Clause 25. The system of any one of clauses 18-24, wherein the first target type of molecular cargo includes a target type of protein, wherein the detection antibodies include detection antibodies configured to bind the target type of protein, and wherein the second target type of molecular cargo includes a target type of RNA, wherein the molecular beacons include molecular beacons configured to bind the target type of RNA.

Clause 26. The system of any one of clauses 18-25, wherein the target type of RNA is selected from microRNA (miRNA), messenger RNA (mRNA), other RNA types, or combinations thereof.

Clause 27. The system of any one of clauses 18-26, wherein the detection antibodies are conjugated with one or more fluorophores for fluorescent imaging.

Clause 28. The system of any one of clauses 18-27, wherein the molecular beacons comprise one or more fluorescent dye sequences for fluorescent imaging.

Clause 29. The system of any one of clauses 18-28, wherein the molecular beacons comprise one or more locked nucleic acid (LNA) nucleotides to improve thermal stability and nuclease resistance.

Clause 30. The system of any one of clauses 18-29, wherein the molecular beacons are selected from any one of SEQ ID NO: 1-13.

Clause 31. The system of any one of clauses 18-30, wherein the fluorescent imaging device is capable of performing total internal reflection fluorescence microscopy (TIRFM).

### EXAMPLES

### Example 1

### Materials

0.01% (w/v) poly-L-lysine (PLL, Millipore Sigma, Burlington, MA), 5 kDa methoxy-poly (ethylene glycol)-succinimidyl valerate (mPEG-SVA) (Thermo Fisher Scientific, Waltham, MA), 0.1 M 4-(2-hydroxyethyl)-1-piperazineethanesulfonic acid (HEPES) buffer (pH=8.5) (Thermo Fisher Scientific), 4-benzoylbenzyl-trimethylammonium chloride (PLPP) (Alvéole, France), NeutrAvidin (NA) (Thermo Fisher Scientific), bovine serum albumin (BSA) (Millipore Sigma), Tris-Ethylenediaminetetraacetic acid (EDTA) buffer (Thermo Fisher Scientific), E. coli (VB200815-1011zys), E. coli (VB200815-1012qpx), E. coli (VB200815-1013ugb) (VectorBuilder Inc., Chicago, IL). All capture and detection antibodies used in the study are provided in Table 1. Capture antibodies were biotinylated using an EZ-Link™ micro Sulfo-NHS-biotinylation kit (Thermo Fisher Scientific). RNA Molecular Beacons (MBs) used in the study are provided in Table 2 (below).

TABLE 1
Antibodies

| Function | Target | Vendor | Catalog No. |
|---|---|---|---|
| Capture Antibody | CD63 | R&D system | MAB5048 |
| | CD9 | R&D system | MAB1880 |
| | EGFR | ImClone LLC | Erbitux-C225 |
| | ARF6 | R&D system | NBP2-41263 |
| | Annexin A1 | R&D system | MAB3770 |
| | IgG | Invitrogen | 31903 |
| Detection Antibody | CD63 Alexa Fluor 488 | Santa Cruz Biotechnology | #sc-5275 |
| | CD9 Alexa Fluor 594 | Santa Cruz Biotechnology | #sc-59140 |
| | CD81 Alexa Fluor 640 | Santa Cruz Biotechnology | #sc-166029 |
| | EGFR Alexa Fluor 561 | Cell Signaling | #5616-D38B1 |

### Substrate Fabrication

Coverslips were cleaned with ethanol and then deionized (DI) water via sonication for 3 min. The surface of the coverslip was treated with oxygen plasma for 1 min to activate the surface. A small drop of 0.01% (w/v) PLL was placed onto parafilm, where the treated coverslip was then placed for an even distribution of the PLL. After incubating the coverslip for 30 min at room temperature (RT), the PLL-coated coverslip was rinsed with DI water and dried with nitrogen flow. Following the same method, 100 mg/ml of mPEG-SVA diluted in 0.1 M HEPES was evenly distributed on the PLL-coated coverslip. The coverslip was incubated at RT for 1 h before rinsing with DI water and drying with a nitrogen airflow. The treated coverslip could be stored for three weeks at 4° C. before use.

### Device Fabrication and Surface Modification

The passivated coverslip was photopatterned using the PRIMO optical module (Alvéole, France) mounted on an automated inverted microscope (Nikon Eclipse Ti Inverted Microscope System, Melville, NY). Briefly, grayscale images were translated into UV light via a DMD that allows for a maskless illumination of different UV intensities correlating to the corresponding grayscale values. Following the passivation of the coverslip, PLPP gel was diluted in 96% ethanol to distribute the gel throughout the surface of the coverslip evenly. After the ethanol evaporated, a silicone spacer (W×L 3.5 mm×3.5 mm, 64 wells, Grace Bio Labs, Bend, OR) was placed on top of the PEG-coated coverslip. A five-by-five array of 20-μm diameter circles spaced 80 μm center-to-center was exposed onto the coverslip with the PRIMO optical module. To optimize the relative fluorescence intensity (RFI) between samples and their controls of the detection probes, microdomains at different grayscale values, including 0, 25, 50, 75, and 95% with UV doses, including 10, 20, and 30 mJ/mm² were examined (see Table 3). After the UV illumination was completed, the photoetched coverslip was washed under a stream of DI water and dried by nitrogen flow. A microscopy slide (Fisher Scientific) was placed under the coverslip, and the 64-well ProPlate microarray system (Grace Bio Labs) was placed gently on the faced-up photoetched coverslip. The assembled array was secured by self-cut Delrin snap clips (Grace Bio Labs) to avoid leakage or potential contamination. The photoetched coverslip was rehydrated with phosphate-buffered saline (PBS) for 15 min before antibody functionalizing of the microdomains.

### Cell Culture

U251 and Gli36 GBM cell lines were cultured in Dulbecco's Modified Eagle Medium (DMEM). SF268, SF295, SF539, SNB19, and SNB-75 GBM cell lines were cultured in Roswell Park Memorial Institute (RPMI) 1640 medium. All cell culture media was prepared with 10% (v/v) fetal bovine serum (FBS) and 1% (v/v) penicillin-streptomycin. Cell lines were first cultured to 90% confluence at 37° C. in a 5% CO₂ incubator. Before EV collection, cells were washed with PBS twice, after which the cells were incubated in media supplemented with 10% (v/v) EV-depleted FBS. The FBS was filtered by tangential flow filtration (TFF) (MWCO: 300 kDa) from which the permeate containing EV-depleted FBS was used. After two days of cell culture, the EV-enriched cell culture medium (CCM) was collected and centrifuged at 2,000×g for 7 min at RT to separate cell debris before further analysis.

### Human Tumor Specimen Collection

Human tumor tissue was obtained under Institutional Review Board (IRB)-approved protocols at MD Anderson Cancer Center (PA 19-0661) in accordance with national guidelines. All patients signed informed consent forms during clinical visits before surgery and sample collection. Patients did not receive compensation in return for their participation in this study.

### Engineered-EV RNA Model System

Cell transfection was conducted via a cellular nanoporation (CNP) biochip. Briefly, a single layer of Gli36 cells (˜ eight million) was spread overnight on a 1 cm×1 cm 3D CNP silicon chip surface. Cel-miR-39, cel-miR-54, and cel-miR-238 plasmids at a weight ratio of 1:1:1 were pre-mixed at a concentration of 100 ng/ml each in PBS for transfection. The plasmid solution was injected into individual cells via nanochannels using a 200 V electric field for a total of 5 pulses, at 10 ms durations and 0.1 s intervals. EVs were collected from the cell supernatant after 24 h of the cell transfection.

### Healthy Donor Serum Collection

10 mL of whole blood from healthy donors was collected into BD Serum Separation Tubes (SST) (Thermo Fisher Scientific). SSTs were gently placed upright to coagulate for 60 min after being rocked 10 times. The SSTs were centrifuged at RT at 1,100×g for 10 min. The serum was stored in 1 mL aliquots at −80° C. All blood samples were collected under an approved Institutional Review Board (IRB) at The Ohio State University (IRB #2018H0268).

### EV Purification

The EV-enriched CCM and healthy donor serum were introduced into a TFF system as described by previous technique to purify EVs. In brief, CCM or serum was circulated through a 500 kDa TFF hollow fiber filter cartridge, where EVs were retained and enriched in the system (˜2 mL), while free proteins and nucleic acids permeated through the filter. Further diafiltration cycles with PBS were performed until pure EVs were obtained (150 mL of PBS in ˜ 80 min). The EVs were further enriched by spinning down the sample within a 10 kDa ultracentrifugal unit at 3000× g at 4° C. until a final volume of 100 μL was achieved.

### EV Size and Concentration Quantification

A tunable resistive pulse sensing (TRPS) method, qNano Gold (Izon Sciences, Boston, MA), was employed to quantify the size and concentration of EVs. 35 μL of the sample was pipetted into NP100 (50-330 nm) and NP600 (275-1570 nm) nanopore membranes. A pressure of 10 mbar and a voltage of 0.48 and 0.26 V was applied for the NP100 and the NP600, respectively. Polystyrene nanoparticles (CPC100 and CPC400) were used to calibrate the samples.

### RNA Molecular Beacon (MB) Design and Quantification

MBs (listed 5′→3′) targeting RNAs detected in this study are listed in Table 2. Locked nucleic acid (LNA) nucleotides (depicted as +) were incorporated into oligonucleotide strands to improve the thermal stability and nuclease resistance of the MBs for incubation at 37° C. The MBs were custom synthesized and purified using high-performance liquid chromatography (HPLC) (Integrated DNA Technologies, Coralville, IA).

TABLE 2
RNA Molecular Beacons

| | | SEQ |
|---|---|---|
| RNA | RNA Molecular Beacon Sequence | ID NO |
| miR-21 | T+CAA+CA/iCy3/+TCA+GT+CT+GATAAGCTAACTTATCAGACTGA/ | 1 |
| | 3BHQ_2/ | |
| miR-21 | T+CAA+CA/iCy5/+TCA+GT+CT+GATAAGCTAACTTATCAGACTGA/ | 2 |
| | 3IAbRQSp/ | |
| AXL-1 | +CTCCC+C/i6-FAMK/+GGA+TTT+GGCA+CTGCAGTGCCAAATCC/ | 3 |
| | 3BHQ_1/ | |
| AXL-2 | +GTG+ATT/iCy5/+CTG+AGC+TGG+CTGACCAAGCCAGCTCAG/ | 4 |
| | 3IAbRQSp/ | |
| AXL-3 | +TGG+TGT/iCy3/+CTA+GTT+AGT+CACAACTGTGTGACTAACTAG/ | 5 |
| | 3BHQ_2/ | |
| miR-9-5p | +CAT+ACA/iCy3/+GCT+AGA+TAACCAAA+GATTGGTTATCTAGC/ | 6 |
| | 3BHQ_2/ | |
| NSF | +GTT+G/iFluorT/C+CCA+CTG+AGA+AGGCCTTCTCAGTGG/3BHQ_1/ | 7 |
| NCAN | +GCT+CCA/iCy3/+GGC+ATA+TCC+ACCTCATGGATATGCC/3BHQ_2/ | 8 |
| miR-1246-5p | +CCT+GC/iCy5/+TC+CAA+AAA+TCCATTCGGATTTTTGGA/3IAbRQSp/ | 9 |
| Cel-miR-39-3p | +CAA+GC/iFluorT/+GAT+TTA+CAC+CCGGTGAGGGTGTAAATC/ | 10 |
| | 3BHQ_1/ | |
| Cel-miR-54-3p | +GTT+CTC/iCy3/+GTC+GTC+TCA+TATCCTGGATATGAGACGAC/ | 11 |
| | 3BHQ_2/ | |
| Cel-miR-238-3p | +CTTT+GAA/iCy5/+CGT+CC+GA+GAACATCCATTCTCGGACG/ | 12 |
| | 3IAbRQSp/ | |
| P53 | +CTCCGT/CY5/CATGTGCTGTGACTTCACAGCACATG/BHQ3-3/ | 13 |

Locked nucleic acid (LNA) nucleotides are depicted as + on the 5′-side, e.g., “+G” indicates a G LNA.
/iCy3/ indicates an internal Cy3 ™ Dye (Freedom ™ Dyes, Integrated DNA Technologies, Inc., “IDT”).
/iCy5/ indicates an internal Cy5 ™ Dye (Freedom ™ Dyes, IDT).
/CY5/ indicates a Cy5 ™ Dye.
/i6-FAMK/ indicates an internal 6-FAM (Azide) Dye (IDT).
/iFluorT/ indicates an internal Fluorescein deoxy thymidine (dT) dye (IDT).
/3IAbRQSp/ indicates a 3′-lowa Black ® red fluorescent quencher (IDT).
/3BHQ_1/ indicates a 3′-Black Hole Quencher ®-1 (IDT).
/3BHQ_2/ indicates a 3′-Black Hole Quencher ®-2 (IDT).
/BHQ3-3/ indicates a 3′-Black Hole Quencher ®-3 (IDT).

### EV Protein and RNA Staining

10 μg/μL of MBs diluted in 1× Tris-EDTA (TE) buffer were mixed with the EV sample for 1 h at 37° C. As for protein detection, 0.4 μg/mL of fluorescently labeled monoclonal antibodies were diluted into a solution of 3% BSA and 0.05% (v/v) Tween® 20 in PBS and were added to the EV sample for 1 h at RT. For single biomarker analysis, sole detection probes were added. To analyze multiple proteins or RNAs, the probes were added sequentially, monoclonal antibodies were added first, followed by MBs.

Single EV Capture using the ^{siEV}PRA

0.1 mg/ml of NA was added to the chip and allowed to physisorb onto the photocleaved microdomains for 30 min. The chip was washed with PBS thoroughly to remove excess nucleic acids. A blocking solution of 3% BSA and 100 mg/ml of mPEG-SVA was added to avoid unwanted non-specific binding. Subsequently, biotinylated anti-CD63 and anti-CD9, anti-EGFR, anti-ARF6, anti-Annexin A1, and anti-IgG were added at 20 μg/mL each and allowed to sit overnight at 4° C. 3% BSA was added for 1 h to further block after the capture antibodies were washed away. EVs were then added and allowed to tether to the antibodies for 2 h at RT. Unbounded EVs were later washed away with PBS.

### Image Analysis

The images of fluorescently labeled siEVs were obtained by TIRFM (Nikon Eclipse Ti Inverted Microscope System, Melville, NY) with a 100× oil immersion lens. An automatic algorithm quantified the TIRFM images by detecting all bright spots by determining the outline of each bright spot as defined by varying fluorescent intensities throughout the image. The background noise was removed using a Wavelet de-noising method, and each bright spot's net signal was obtained. The sum of all the bright spots within each microdomain was employed to calculate the total fluorescence intensity of the sample alongside a statistical distribution of the mean fluorescent intensity. The total fluorescence intensity of samples was normalized to the total fluorescence intensity of negative controls as relative fluorescence intensities (RFI).

### Enzyme-linked Immunosorbent Assay (ELISA)

Epidermal growth factor receptor (EGFR) protein expression levels on the surface of Gli36-derived EVs were quantified using an EGFR Human ELISA kit (Thermo Fisher Scientific). EVs were spiked in healthy donor serum at different concentrations ranging from 0 to 1.0×10¹¹ particles/mL while maintaining the serum-derived EV concentration at 1.0×10⁹ particles/mL. EGFR expression was quantified according to the manufacturer's instructions.

qRT-PCR

Cel-miR-39-3p levels within the engineered EVs were quantified using qRT-PCR. Total RNA from the cells and EVs was isolated and purified using a RNeasy Mini Kit and a miRNeasy Serum/Plasma kit (Qiagen, Hilden, Germany), respectively, according to the manufacturer's instructions. cDNA was synthesized from the total RNA using a High-Capacity cDNA Reverse Transcription kit (Applied Biosystems, Foster City, CA) on a thermal cycler (Veriti 96-Well Thermal Cycler, Applied Biosystems). Cel-miR-39-3p expression was quantified using a TaqMan Gene Expression assay (Thermo Fisher Scientific, Assay Id: Hs01125301_m1) on a Real-Time PCR System (Applied Biosystems, Thermo Fisher Scientific).

### Scanning Electron Microscopy (SEM)

TFF-purified EVs were tethered to the micropatterned coverslip overnight at 4° C. The tethered EVs were fixed in a 2% glutaraldehyde (Millipore Sigma) and 0.1 M sodium cacodylate solution (Electron Microscopy Sciences, Hatfield, PA) for 3 hr. EVs were incubated in 1% osmium tetraoxide (Electron Microscopy Sciences) and 0.1 M sodium cacodylate for 2 h after washing with a 0.1 M sodium cacodylate solution. Subsequently, the sample was dehydrated with increasing ethanol concentrations (50, 70, 85, 95, and 100%) for 30 min each. Later, the CO₂ critical point dryer (Tousimis, Rockville, MD) was applied to dry the sample. Last, a ˜2 nm layer of gold coating was completed using a sputtering machine (Leica EM ACE 600, Buffalo Grove, IL) and was imaged using an SEM (Apreo 2, FEI, Thermo Fisher Scientific).

### RNA Sequencing

RNA, including miRNA, was isolated from cells and cell-derived EVs using the miRNeasy kit (Qiagen). The RNA was eluted with 50 μL of nuclease-free H₂O, and the quality was assessed using an RNA (Pico) chip on an Agilent 2100 Bioanalyzer (Agilent Technologies, Santa Clara, CA). A small RNA sequencing library construction method that utilizes adapters with four degenerated bases to reduce adapter-RNA ligation bias was used to characterize the miRNA (PMID: 29388143). Size selection was performed using a Pippin HT automated size-selection instrument (Sage Science, Beverly, MA), and library concentrations were measured with the NEBNext Library Quant Kit (New England Biolabs, Ipswich, MA). The libraries were pooled to a final concentration of 2 nM and run on a NextSeq sequencer (Illumina, San Diego, CA). The small RNA sequencing (sRNA-Seq) data was analyzed with sRNAnalyzer. The quantity of miRNA was determined based on the number of mapped reads that were normalized with Count Per Mapped Million (CPM). RNA from cells and EVs were analyzed using Agilent Human Whole Genome 8×60 microarrays with fluorescent probes prepared from isolated RNA samples using Agilent QuickAmp Labeling Kit according to the manufacturer's instructions (Santa Clara, CA). Gene expression information was obtained with Agilent's Feature Extractor and processed with the in-house SLIM pipeline.

### Colocalization Efficiency

An open-source plugin for ImageJ called EzColocalization was employed to visualize and measure the colocalization of EV biomarkers from acquired TIRFM images.

### Statistical Analysis

Data are expressed as the mean±SD. A significant test between different mean values was evaluated using the JMP Pro 14 software (JMP, Cary, NC). Differences between samples were considered statistically significant for p<0.05.

### Example 2

Single-EV Analysis with the ^{siEV}PRA

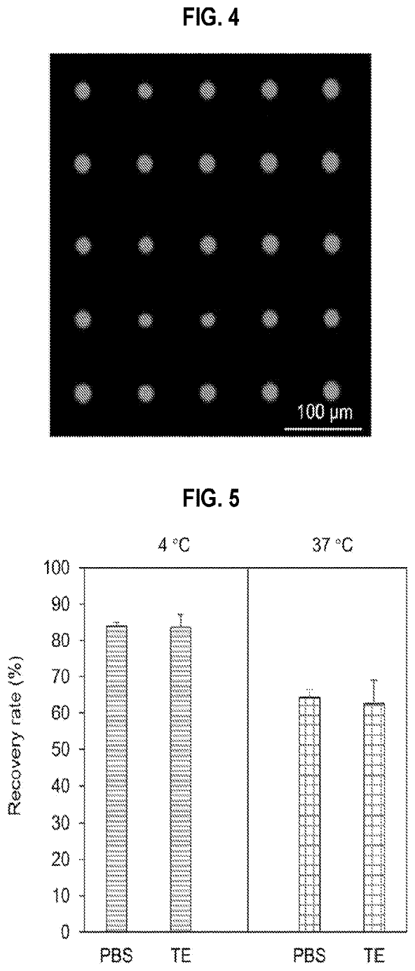

The device was fabricated with the PRIMO optical module (FIG. 3A). A glass coverslip was coated with poly-L-lysine (PLL) through physisorption and methoxy-poly(ethylene glycol)-succinimidyl valerate (mPEG-SVA) was covalently bound to the surface through N-hydroxysuccinimide (NHS) chemistry creating a non-biofouling coating. A five-by-five array of 20-μm diameter circles was cleaved from the mPEG monolayer via UV projections translated by a DMD in the presence of 4-benzoylbenzyl-trimethylammonium chloride (PLPP) as a photoactivator. The level of photoscission correlates to both the grayscale value of a digital template and the UV dose. A 50% grayscale value and a 20 mJ/mm² dose were selected as they rendered the highest relative fluorescent intensity (RFI) to capture siEVs relative to the control (phosphate buffered saline, PBS) and minimized non-specific binding within the microdomains (Table 3). The optimized grayscale value and dose demonstrated homogenous adsorption of NeutrAvidin (NA) with specificity to the photocleaved surfaces (FIG. 4). Given the uniformity of the NA layer, biotinylated antibodies against CD63 and CD9, epidermal growth factor receptor (EGFR), ARF6, and Annexin A1, which are present as membrane proteins on EVs, were patterned in the microdomains to tether siEVs selectively (FIG. 3A).

TABLE 3
RFI Optimization by Controlling Monolayer Degradation

| | |
|---|---|
| Dose | Gray Scale |

| (mJ/mm²) | 100% | 95% | 75% | 50% | 25% | 0% |
|---|---|---|---|---|---|---|
| 30 | 1 | 1.5 | 4 | 2.5 | 1 | 1 |
| 20 | 1 | 1 | 3.5 | 6 | 1 | 1 |
| 10 | 1 | 1 | 1.5 | 3.5 | 1 | 1 |

To test the presence of siEVs on the microdomains, a fluorescently labeled antibody against CD63 and an MB targeting miR-21, an abundant EV-enveloped miRNA, was used as detection probes and visualized via TIRFM. Each green fluorescent spot represented a siEV expressing CD63, and each red fluorescent spot represented a siEV carrying miR-21. Each yellow spot demonstrated the colocalization of both biomarkers at a singlevesicle resolution. Conversely, fluorescent signals in control samples were significantly lower, indicating the ability of the ^{siEV}PRA to multiplex different biomolecule species in siEVs (FIG. 3D). Furthermore, acquired TIRFM images could be quantified as statistical fluorescent signal distributions to analyze biomarker expression on siEVs (FIG. 3E) or to quantify the RFI of the sample (FIG. 3F), which revealed an RFI of 12.16±0.50 and 11.26±0.08 for siEVs relative to the control samples (ANOVA, p<0.0001 for CD63 and miR-21). After EV immobilization, scanning electron microscopy (SEM) was performed on the device to further validate the fluorescent signals observed on the microdomains as originating from siEVs. The SEM images revealed single, round vesicles, confirming the presence of siEVs tethered on the microdomains (FIG. 3G). TRPS measurements on the EV samples used for the ^{siEV}PRA demonstrated a mean-siEV diameter of 150 nm, consistent with the size of the vesicles observed by SEM (FIG. 3H). Thus, the ^{siEV}PRA successfully captures siEVs in distinct surface array positions and multiplexes protein and RNA signals via immunoaffinity and MB RNA hybridization, respectively.

Specificity and Sensitivity of RNA and Protein Detection in siEVs

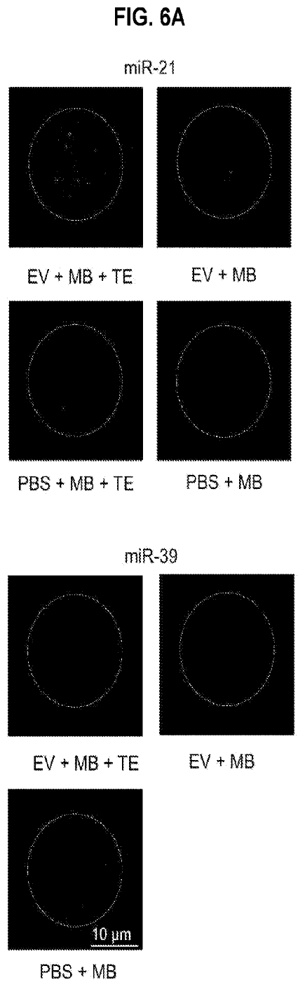

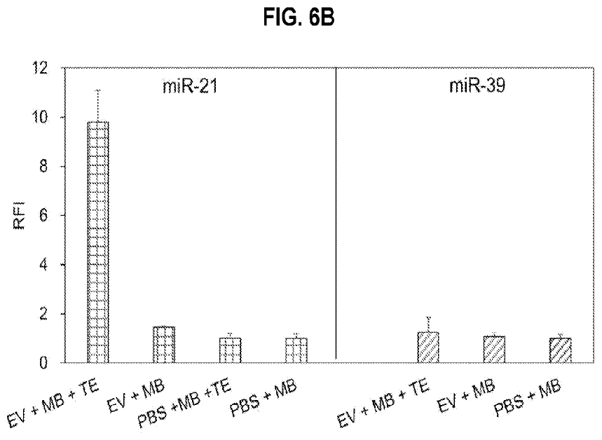

Although there are methods available to detect proteins on siEVs, detecting RNA without altering or damaging the integrity of the vesicles remains a challenge. To detect membrane-enveloped RNAs, MBs were diluted in a tris-EDTA (TE) buffer to partially permeate the lipid bilayer of the EVs, allowing the MBs to penetrate the membrane and hybridize with the desired RNA sequences. The changes in EV concentration when incubating in the TE buffer and PBS were negligible (ANOVA, p=0.65), implying the extent of permeabilization by the TE buffer was not detrimental to the integrity of the EVs (FIG. 5). To ensure the specificity of the MBs to the desired RNA targets on the ^{siEV}PRA, miR-21, a human miRNA, and miR-39, a non-human miRNA abundant in Caenorhabditis elegans, were tested in siEVs derived from Gli36 cells, a human glioma cell line. Gli36-derived EVs detected with MBs targeting miR-21 exhibited single fluorescent spots within the microdomain when diluted in the TE buffer (FIG. 6A). The MB formulation diluted in the TE buffer produced a fluorescent signal 6.30±0.50 times higher than the formulation diluted in PBS when applied to the immobilized siEVs (ANOVA, p<0.0001), indicating the necessity for partial permeabilization. Furthermore, the siEV signals obtained from partial permeabilization were 9.65±1.28 times higher than MBs diluted solely in the TE buffer (ANOVA, p=0.0054) and 9.80±1.30 times higher than MBs diluited solely in PBS (ANOVA, p<0.0001), ensuring the specificity of the MBs to detect RNAs in siEVs. In contrast, Gli36-derived EVs detected with MBs targeting non-human miR-39 within the TE buffer and PBS demonstrated a negligible difference when compared to their respective controls (ANOVA, p=0.62 for TE and p=0.68 for PBS), thus proving the ability of the ^{siEV}PRA to target specific RNA sequences (FIG. 6B).

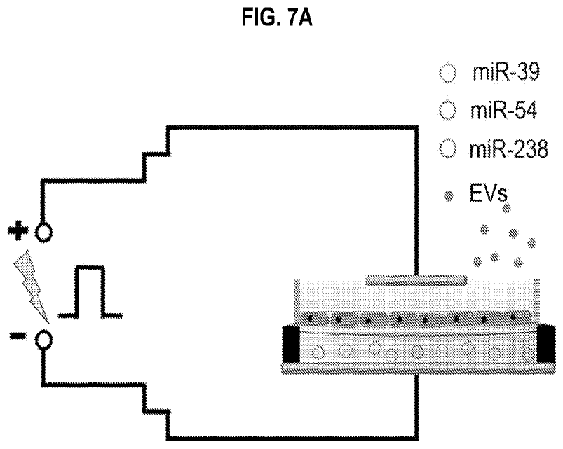

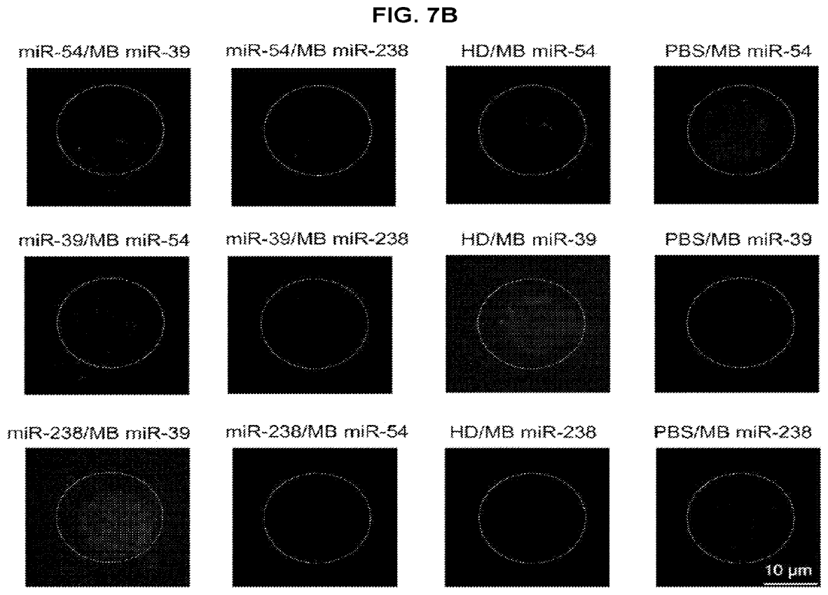

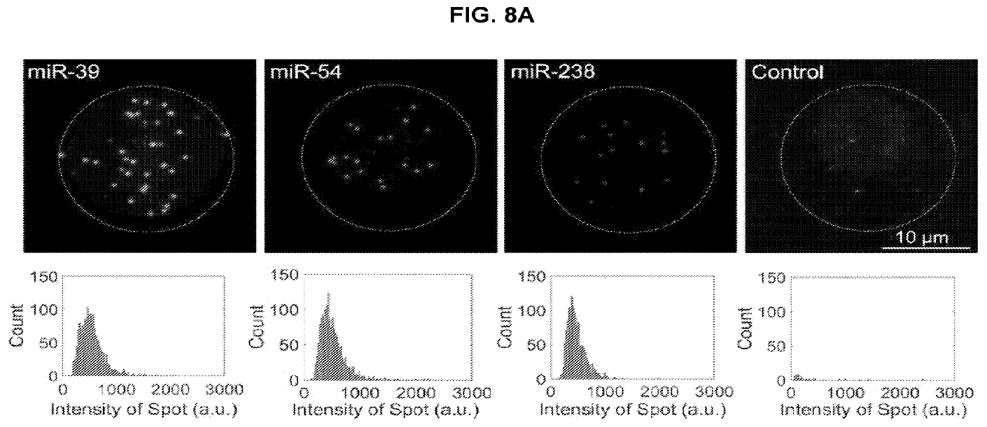

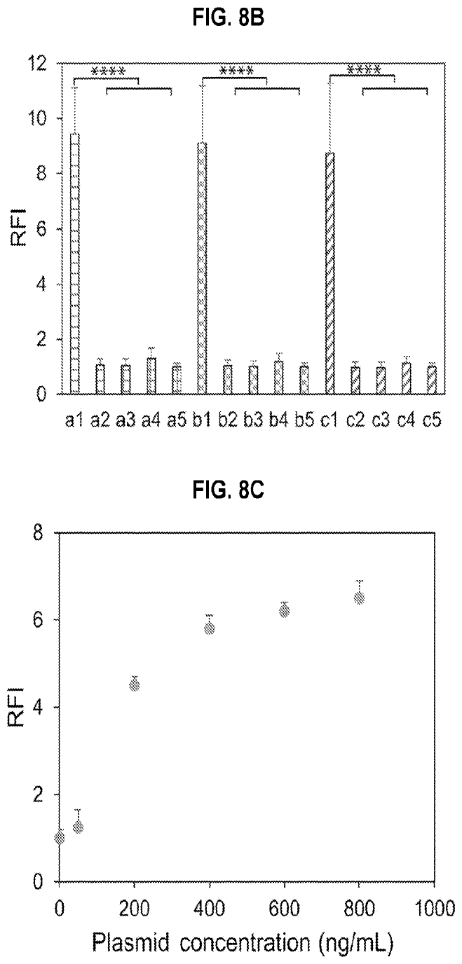

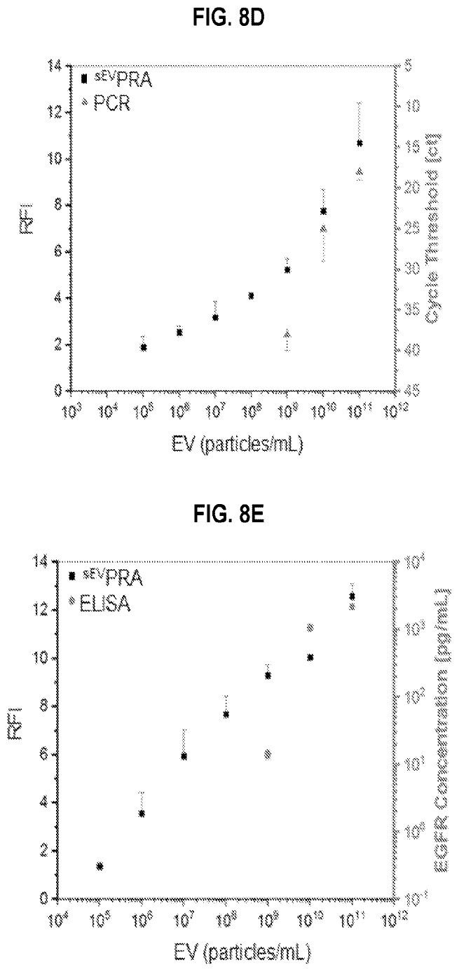

To evaluate the robustness of RNA specificity using the ^{siEV}PRA, Gli36 cells were transfected via electroporation with cel-miR-54, cel-miR-39, and cel-miR-238 plasmids, which are non-human miRNAs (FIG. 7A). EVs harvested from the transfected cells were then detected with MBs targeting miR-39, miR-54, and miR-238. The engineered siEVs loaded with non-human miRNAs were successfully detected as single fluorescent spots within the microdomains when the MBs targeted the corresponding miRNA. In contrast, control samples showed a negligible number of fluorescent spots (FIG. 8A). To ascertain a lack of cross-reactivity between the MBs and the other non-human miRNA, the three different engineered EVs were tested against all the MBs targeting the non-human miRNA. Only the MBs targeting the corresponding nonhuman miRNA loaded within the engineered EVs could be detected, whereas all disparate MBs presented a background level of fluorescent spots (FIG. 7B). Similarly, EVs collected from healthy donor serum presented few fluorescent spots (FIG. 7B). Specifically, miR-54-enriched EVs detected by MBs targeting miR-54 produced a fluorescent signal 9.43±1.68 times higher than the average of the controls (ANOVA, p<0.0001), miR-39-enriched EVs detected by MBs targeting miR-39 produced a fluorescent signal 9.10±2.07 times higher than the average of the controls (ANOVA, p<0.0001), and miR-238-enriched EVs detected by MBs targeting miR-238 produced a fluorescent signal 8.73±2.52 times higher than the average of the controls (ANOVA, p<0.0001) (FIG. 8B). Furthermore, the ^{siEV}PRA was capable of discriminating between the EVs transfected with varying plasmid concentrations, demonstrating the sensitivity of the assay to quantify nucleic acid concentrations within siEVs (FIG. 8C).

The sensitivity of the ^{siEV}PRA for RNA detection in siEVs was compared to conventional bulk PCR. EVs harvested from Gli36 cells loaded with 400 ng/ml of the cel-miR-39 plasmid were diluted serially and detected with the ^{siEV}PRA and PCR for miR-39.

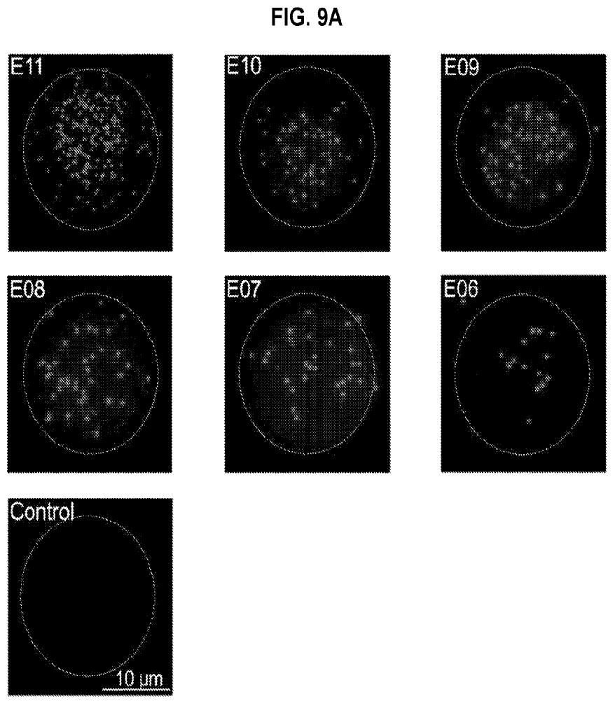

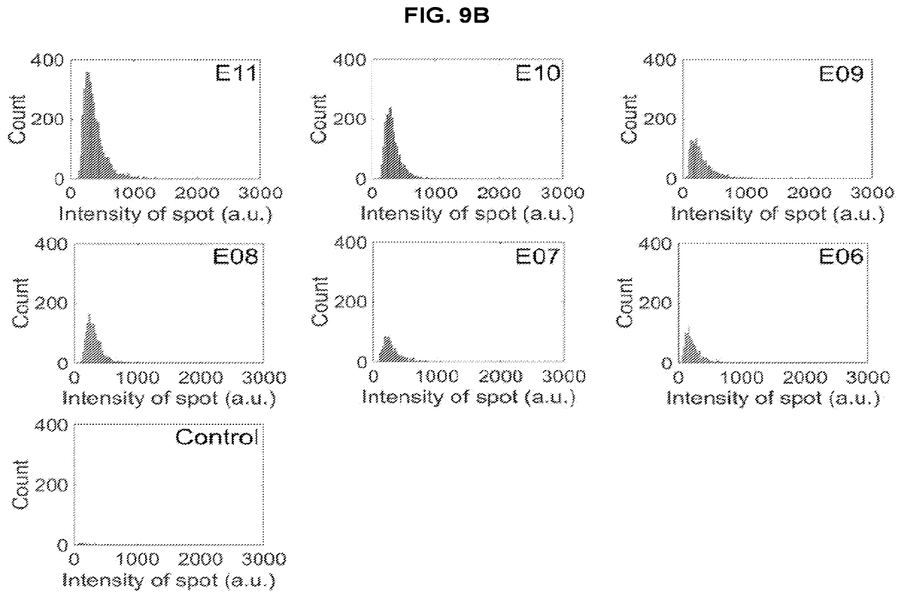

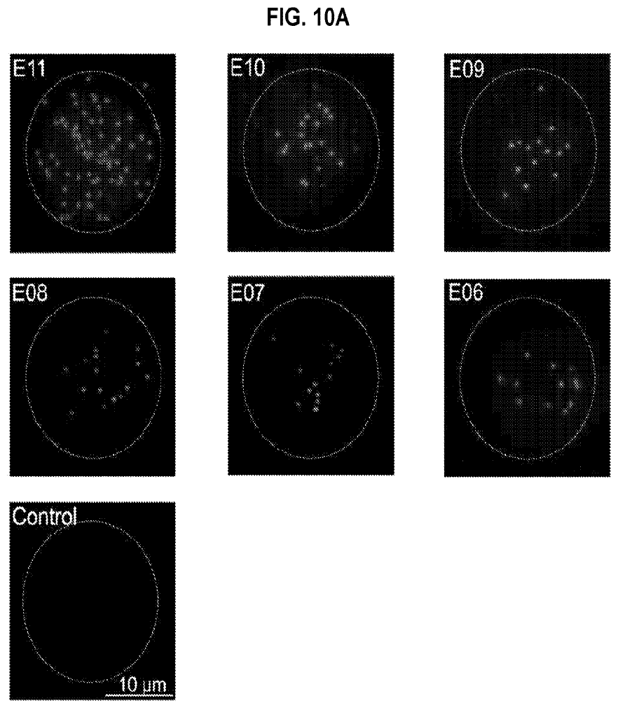

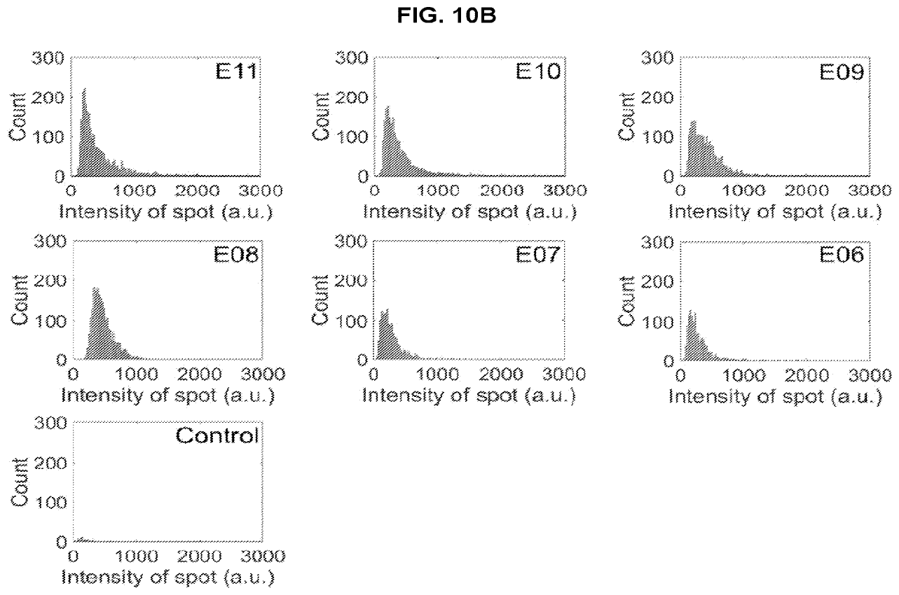

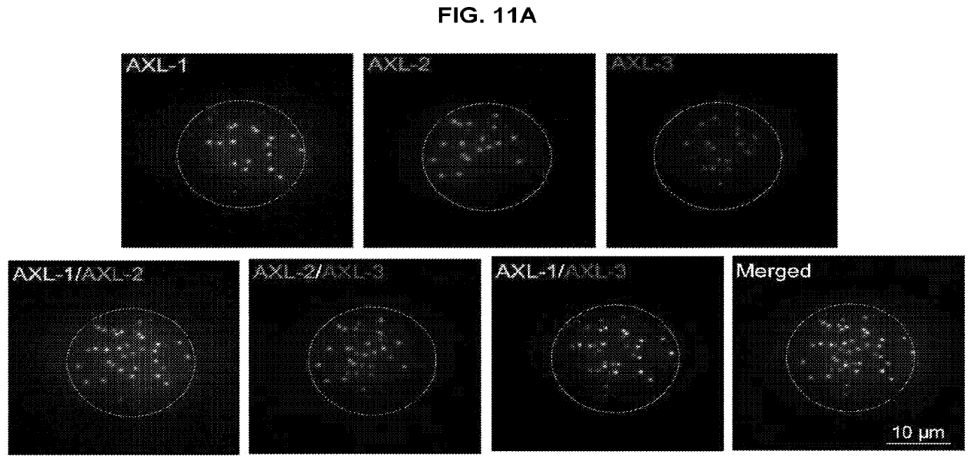

Signal from the sample was detected at a concentration of 1.0×10⁶ vesicles/mL with the ^{siEV}PRA (ANOVA, p=0.01), outperforming the detection of miR-39 with PCR, which was undetectable below a concentration of 1.0×10⁹ vesicles/mL (FIG. 8D; FIG. 9A-B). Moreover, the sensitivity of the ^{siEV}PRA for protein detection in siEVs was compared to standard bulk ELISA. Gli36-derived EVs were diluted serially and detected with both methods for EGFR, a protein upregulated in GBM. Signal from the sample was detected at a concentration of 1.0×10⁶ vesicles/mL with the ^{siEV}PRA (ANOVA, p=0.01), whereas ELISA could not detect EGFR below a concentration of 1.0×10⁹ vesicles/mL (FIG. 2E; FIG. 10A-B). Thus, the ability of the ^{siEV}PRA to outperform conventional bulk analysis methods to detect vesicular RNA and protein, while preserving siEV integrity, indicate its potential for the molecular characterization of EV heterogeneity at minimal concentrations.

### Simultaneous Detection of Various Biomolecule Species

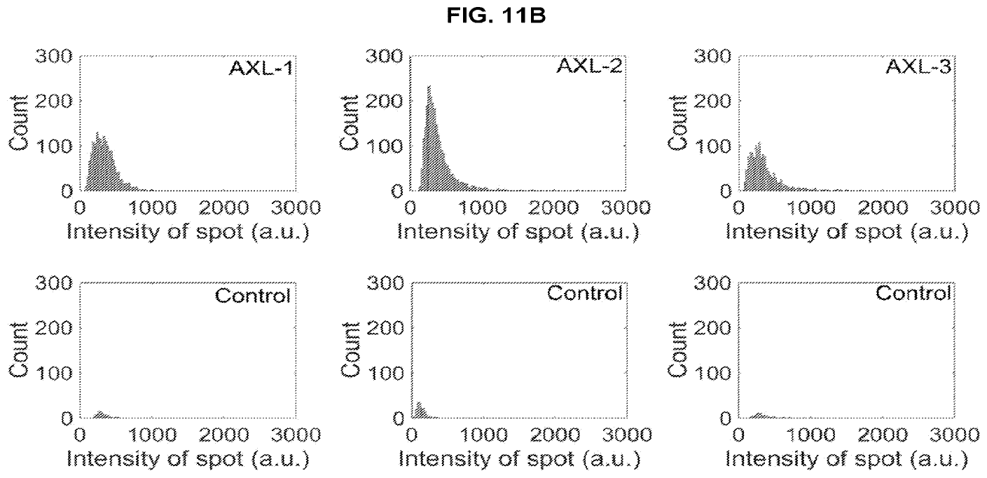

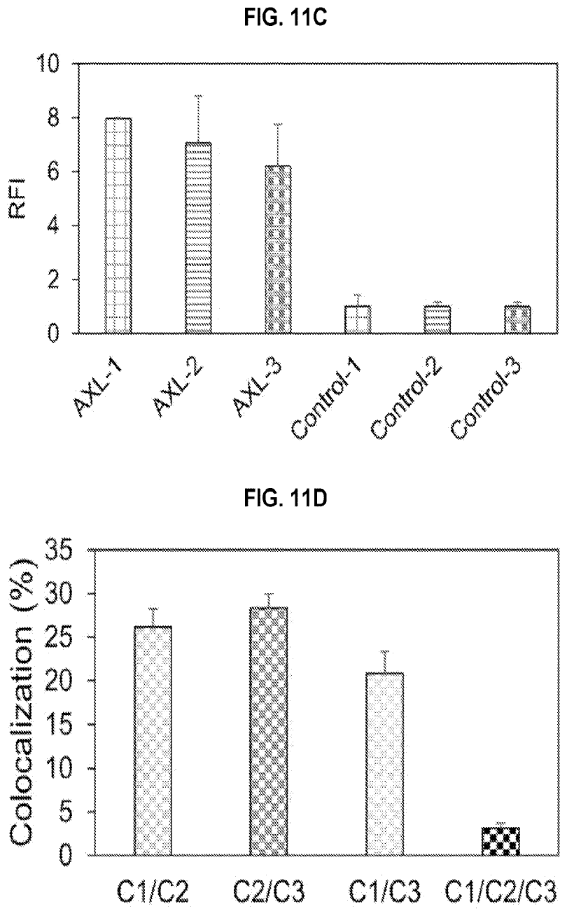

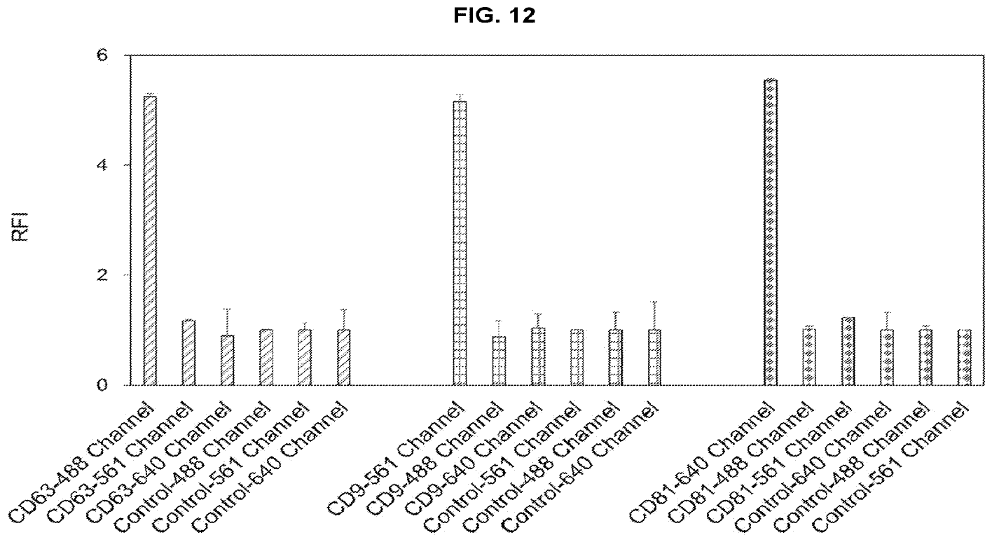

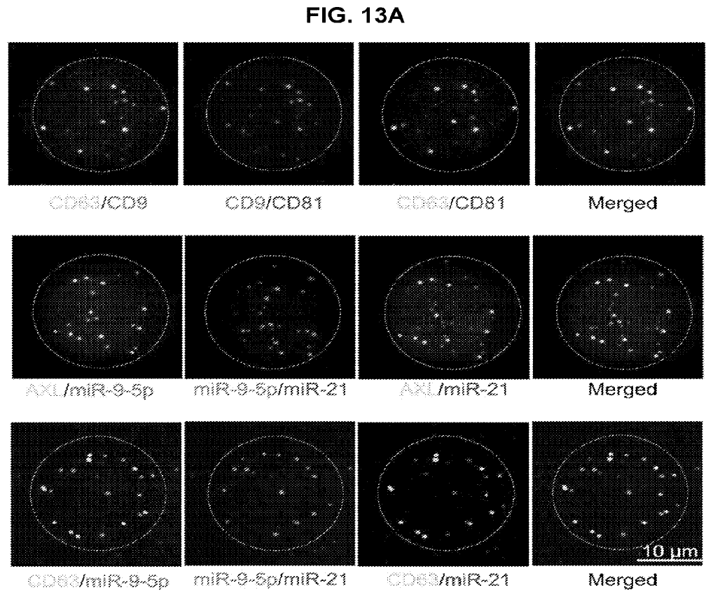

To first determine the ability of the ^{siEV}PRA to multiplex various probes at the single vesicle level, different regions of an mRNA were detected simultaneously within siEVs. Given the length of mRNA strands, three MBs targeting three different regions of the AXL receptor tyrosine kinase (AXL) mRNA, an abundant mRNA found in GBM, were designed such that each MB emitted a different fluorescent signal when hybridized. All three regions of the AXL mRNA were detected in siEVs as single fluorescent spots. Furthermore, magenta, cyan, and yellow spots illustrated the colocalization of two detection probes, whereas white spots demonstrated the colocalization of all detection probes (FIG. 11A). The probability distributions of the single A×L regions detected by the MBs were similar (FIG. 11B). Moreover, the fluorescent intensities of the three different regions on the AXL mRNA showed negligibly different fluorescent intensities (ANOVA, p=0.95), demonstrating a uniform and noncompetitive affinity of the MBs to the different regions of the mRNA strand (FIG. 11C). The colocalization efficiencies for AXL-1 and AXL-2, AXL-2 and AXL-3, AXL-1, and AXL-3, and all three regions were 26.15±2.09%, 28.31±1.59%, 22.84±2.52%, and 3.12±0.58%, respectively (FIG. 11D). Given the homogeneity of the MB hybridization to the different regions of the AXL mRNA, the statistically equal colocalization efficiencies for the simultaneous hybridization of two regions (ANOVA, p=0.69) implies a similar probability for two probes to co-detect RNA within siEVs. Furthermore, the fluorophores were only excited when matched by their corresponding emission (FIG. 12), ensuring the validity of the colocalization as originating from the EV-detecting probes.

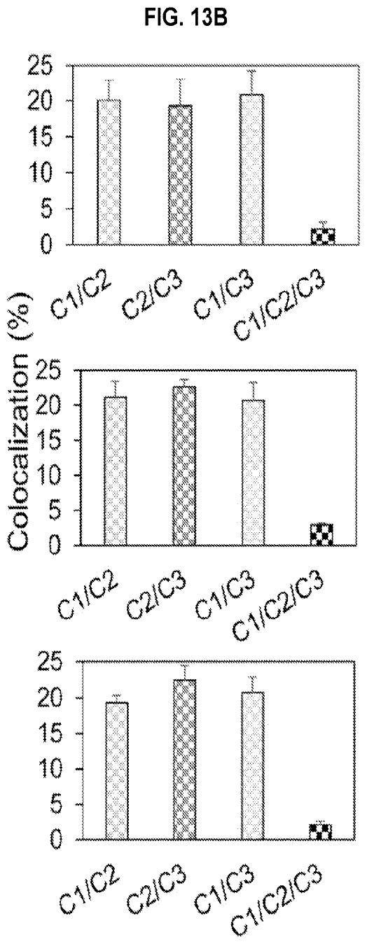

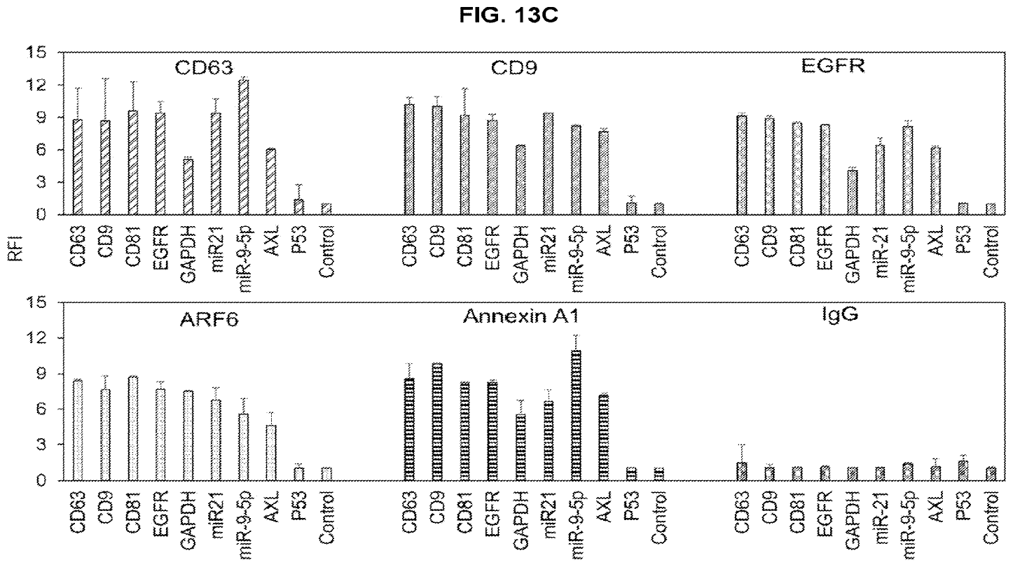

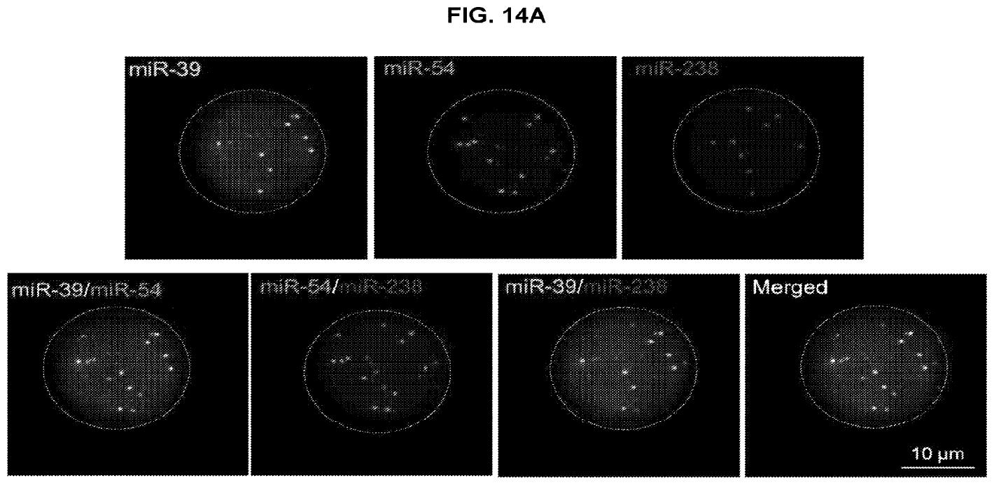

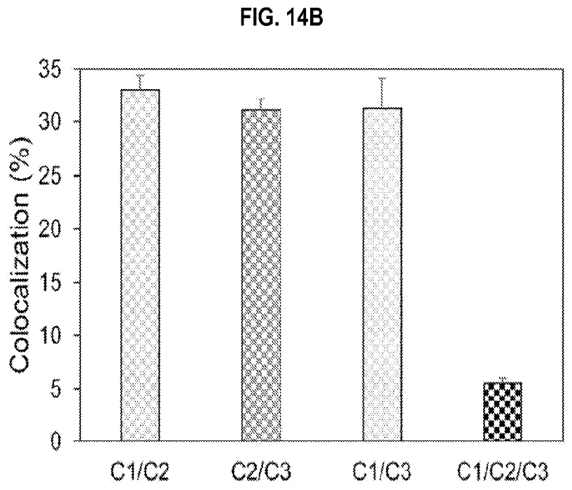

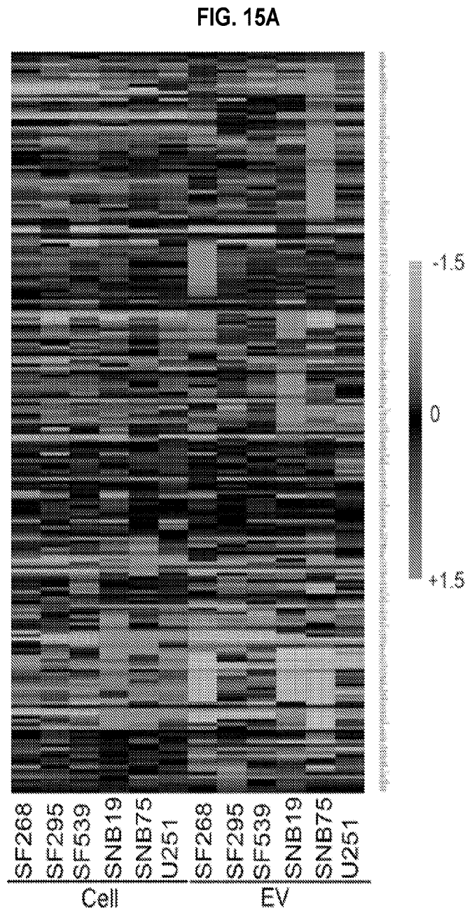

To further test the ability of the ^{siEV}PRA for multiplexed biomarker detection in siEVs, several combinations of proteins and RNAs were screened. CD63, CD81, and CD9 were detected on Gli36-derived EVs (FIG. 13A). The colocalization efficiencies for CD63 and CD9, CD81 and CD9, CD63 and CD81, and all three proteins were 20.08±2.09%, 19.31±1.59%, 20.84±2.52%, and 2.16±0.58%, respectively (FIG. 13B). EVs harvested from Gli36 cells transfected with cel-miR-39, cel-miR-54, and cel-miR-238 plasmids were detected by their respective MBs (FIG. 14A). The colocalization efficiencies for miR-39 and miR-54, miR-54 and miR-238, miR-238 and miR-39, and all miRNA were 32.94±1.47%, 31.10±1.03%, 31.26±2.90%, and 5.51±0.51%, respectively (FIG. 14B). Moreover, proteins and RNAs across various species were detected with the ^{siEV}PRA. First, miR-21, miR-9-5p, and AXL were detected in single Gli36-derived EVs to detect different RNA species, including miRNA and mRNA (FIG. 13A). The colocalization efficiencies for AXL and miR-9-5p, miR-21 and miR-9-5p, AXL and miR-21, and all three RNA biomarkers were 21.15±2.29%, 22.62±1.08%, 20.67±2.58%, and 2.95±0.18%, respectively (FIG. 13B). Second, CD63, miR-21, and miR-9-5p were detected in single Gli36-derived EVs to multiplex proteins and RNA simultaneously (FIG. 13A). The colocalization efficiencies for CD63 and miR-9-5p, miR-21 and miR-9-5p, miR-21 and CD63, and all three biomarkers were 19.30±1.05%, 22.52±1.90%, 20.71±2.23%, and 2.12±0.48%, respectively (FIG. 13B).

The ^{siEV}PRA platform also enabled the sorting and characterization of siEV subpopulations based on different surface proteins by using different antibodies to capture siEVs and subsequently measure their protein and RNA content. Tetraspanins, ARF6, and Annexin A1 are well-known surface proteins expressed in EVs. Nine different biomarkers, including four proteins and five different RNAs, were quantified and revealed different expression levels in siEVs by TIRFM. Fluorescent signals from siEVs showed that expression levels for CD81, CD63, CD9, and EGFR were higher than the other biomarkers independent of the EV subpopulation analyzed. More variability was observed for the different RNAs tested for which there was no clear trend in the level of expression based on the subpopulation analyzed. Thus, these results confirm the heterogeneity in the different EV subpopulations captured on the device (FIG. 13C).

### Single-EV Analysis of RNA Biomarkers in EV Subpopulations and Clinical Samples

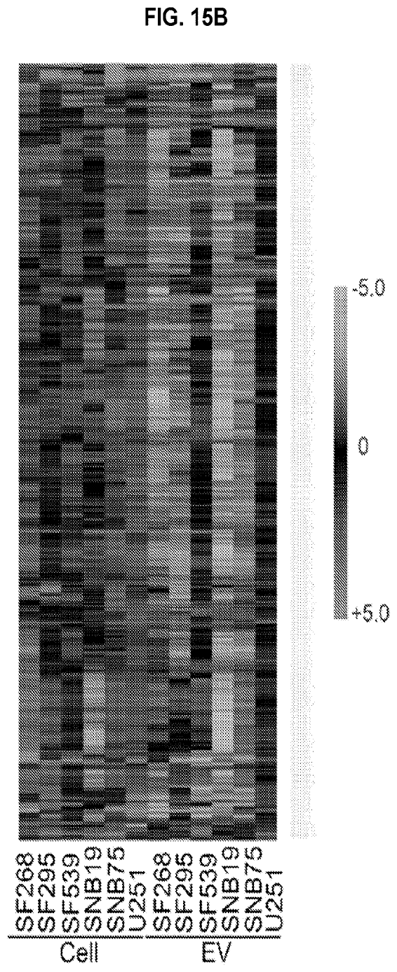

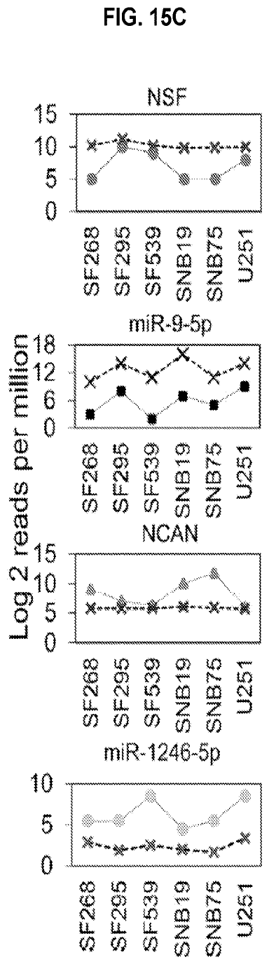

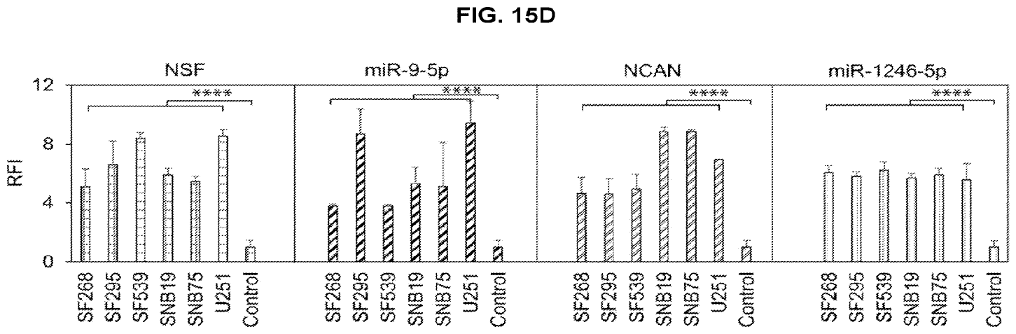

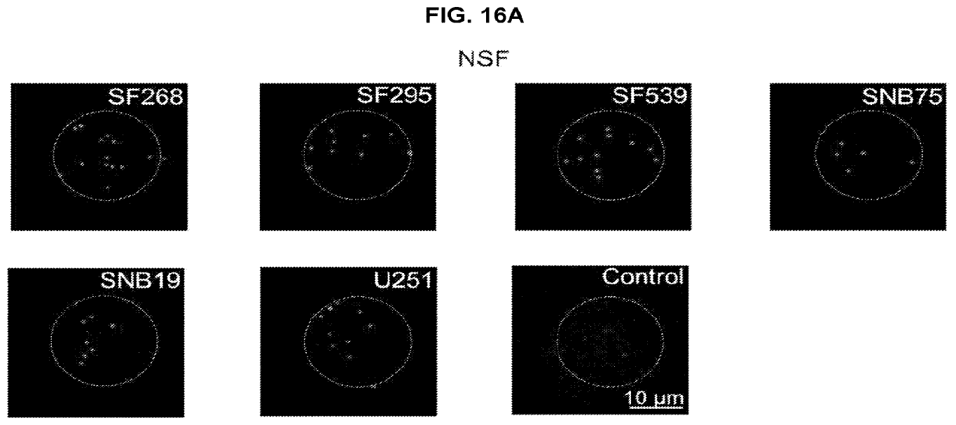

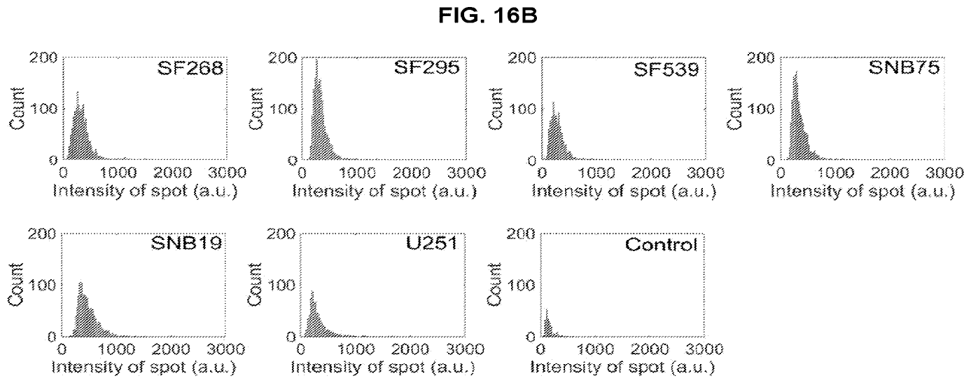

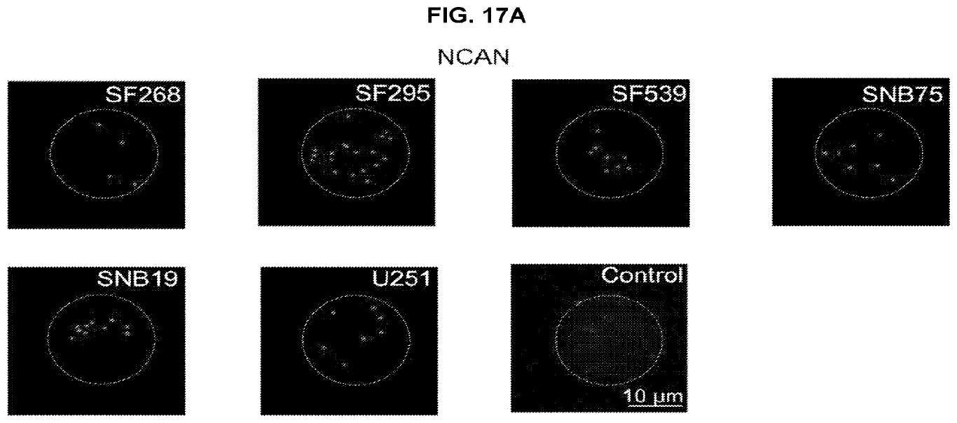

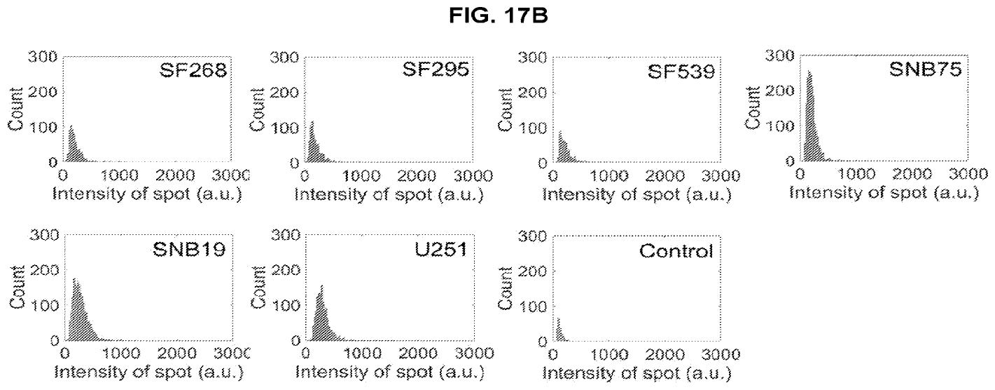

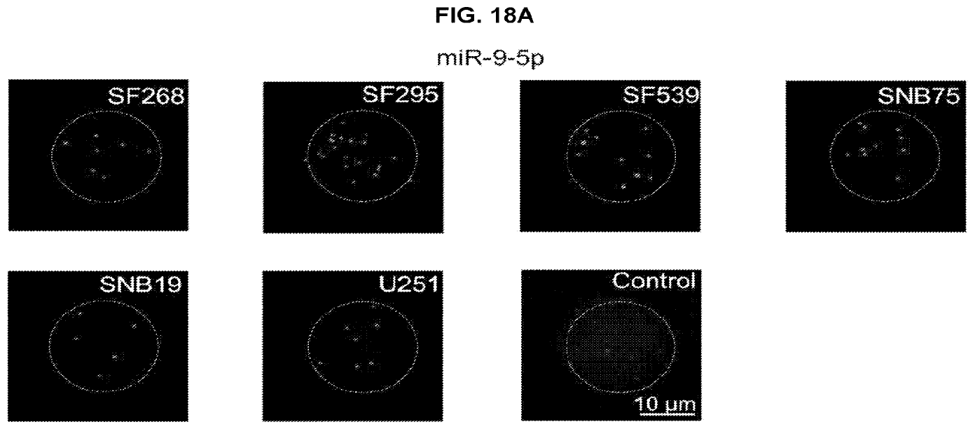

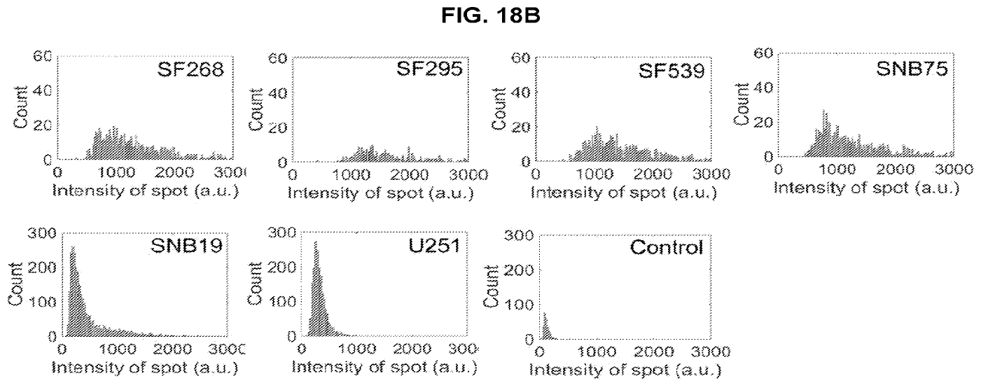

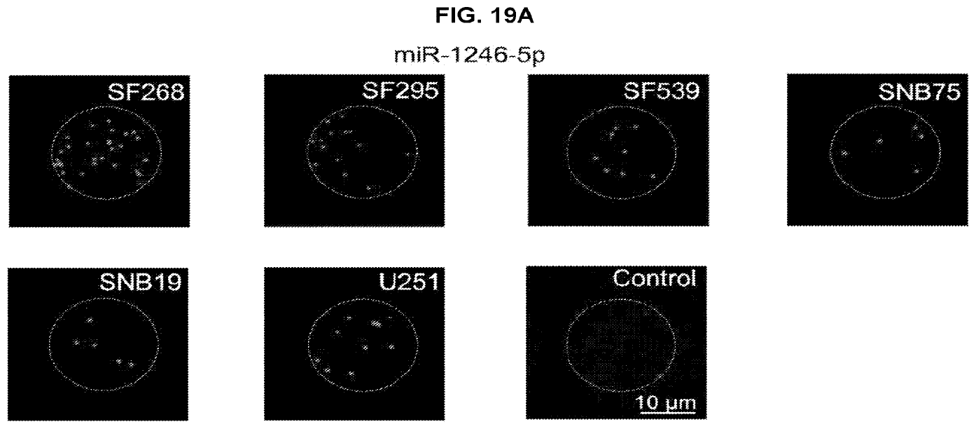

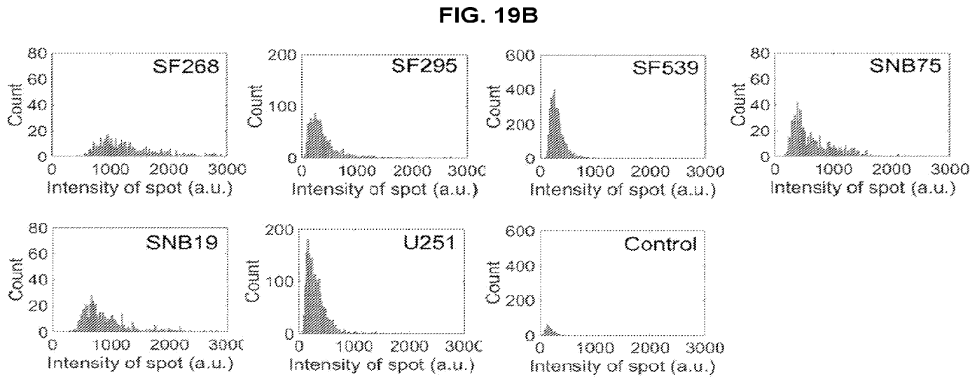

To validate the use of the ^{siEV}PRA for characterizing EV heterogeneity, transcriptomic analysis was performed on six different GBM cell lines and their corresponding EVs, including SF268, SF295, SF539, SNB19, SNB75, and U251 using microarray and small RNA sequencing. Several RNAs were found that exhibited high concentrations in cells and EVs (FIG. 15A). Among them, four transcripts, two mRNAs (NSF and NCAN) and two miRNAs (miR-9-5p and miR-1246-5p) were selected for further analysis since they have also been reported to be associated with GBM. The concentrations of the four selected transcripts measured in the different GBM cell lines showed less variability than their corresponding EVs (FIG. 15B). The heterogeneity of these transcripts in EVs was further explored with the ^{siEV}PRA. NSF, NCAN, miR-9-5p, and miR-1246-5p were measured in siEVs (FIG. 15C; FIG. 16-19). siEVs from the six cell lines exhibited higher RFIs across the four biomarkers than the control samples (ANOVA, p<0.0001). Although the four RNAs were detectable in siEVs, variations in fluorescent signal across EVs from the different cell lines demonstrated vesicular heterogeneity. The statistical distributions for siEV intensity illustrate a more homogeneous expression for the mRNAs compared to the miRNAs. For NSF and NCAN, the distribution maxima were relatively consistent across the EVs from the six cell lines at 356.09±64.20 and 300.14±78.02, respectively (FIG. 16-17). On the other hand, miR-9-5p from SF268-, SF295-, SF539-, and SNB75-derived siEVs had more heterogeneous profiles with distribution maxima shifted to the right; similarly, miR-1246-5p from SF268-, SNB75-, and SNB19-derived siEVs also demonstrated a heterogeneous expression with distribution maxima shifted to the right (FIG. 18). In general, distribution maxima for the miRNA had more variability. Specifically, miR-1246-5p showed more significant discrepancies than other RNAs (FIG. 19). Distribution maxima among all controls showed less variability with values at 149.15±28.92.

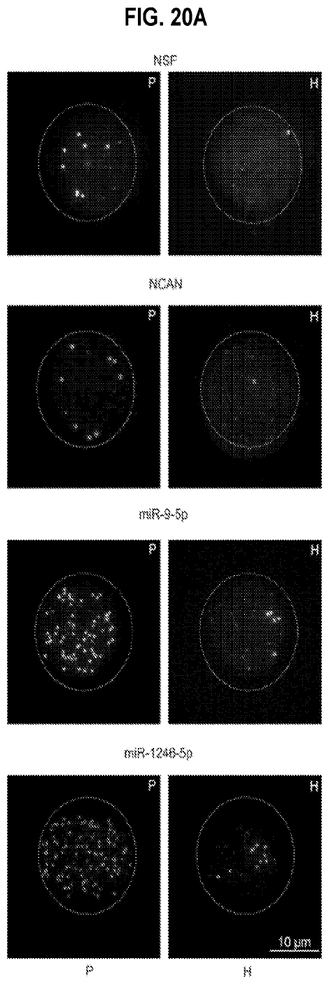

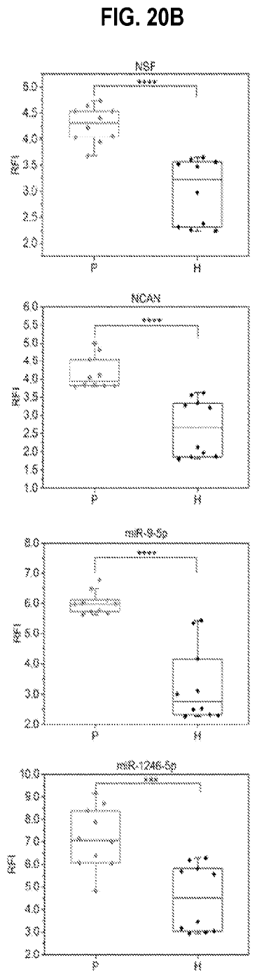

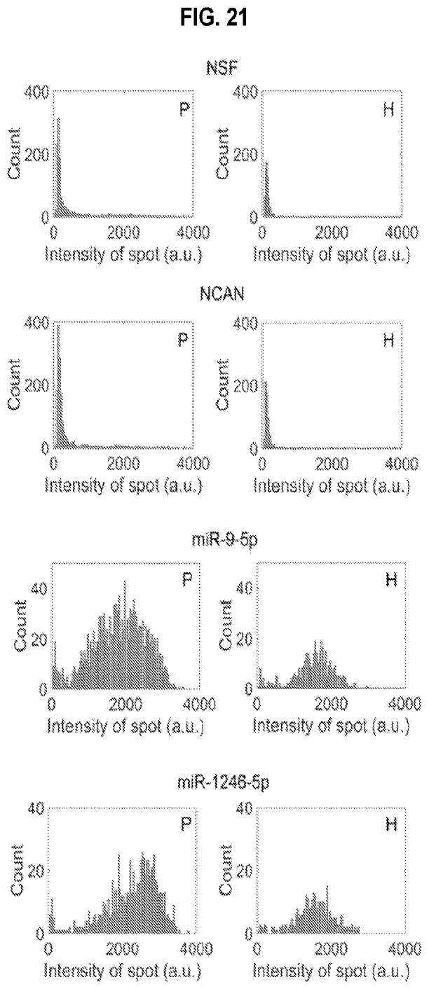

Finally, the ^{siEV}PRA was used to characterize EV subpopulations from GBM patient serum. An average of 20 μL of purified serum was processed from GBM patients at different stages of treatment (n=10). Serum from healthy individuals was also processed as healthy controls (n=10). For the GBM patient samples, fluorescent signals were measured for NSF, NCAN, miR-9-5p, and miR-1246-5p RNAs in siEVs, while fluorescent signals in EVs from healthy donor serum were significantly lower (p<0.0001, FIG. 20A-B). Comparisons of the statistical distributions of siEV intensity for the different RNA species revealed that NSF and NCAN mRNAs presented more homogeneous fluorescent signals with distribution maxima at 355.80±2.76 and 383.47±28.92. On the other hand, the statistical distributions for miR-9-5p and miR-1246-5p miRNAs showed a broad distribution of fluorescent signals with distribution maxima at 1482.67±32.16 and 1136.06±27.43, respectively. However, the statistical distributions of the different RNAs measured from healthy donor serum exhibited less variability, with distribution maxima at 210.16±32.76. Similar trends were observed with the control samples (FIG. 21). These findings confirm the ability of the ^{siEV}PRA to measure RNA heterogeneity in siEVs from complex biofluids. The success of this work opens the possibility for its application in liquid biopsies for cancer diagnoses and prognoses.

The physical and biological heterogeneity that EVs exhibit has made their accurate molecular quantification a difficult task. Current methods for the molecular analysis of EVs, including WB, ELISA, PCR, NGS, and MS, require a high concentration of EVs and a breakdown of their vesicular structure to access their cargo. As a result, crucial molecular information of tissue-specific or disease-specific EVs is lost. To overcome these limitations, several new technologies have been developed to isolate and characterize EVs in situ. However, these platforms still require many EVs per measurement and are limited in characterizing different types of molecular cargo. The siEVPRA was developed as a promising technology that enables the robust investigation of heterogeneity in siEV cargo. The siEVPRA was built as an array of microdomains on a polymer-coated glass surface fabricated by maskless UV photopatterning. Antibodies immobilized within the arrayed surface targeted siEV subpopulations that were detected in situ with fluorescently labeled antibodies and RNA-targeting MBs. 20 μL of complex biofluids (e.g., cell culture media (CCM), serum) is enough to perform a multiparametric characterization of various proteins and RNAs in siEVs to investigate vesicular heterogeneity.

The higher sensitivity of the ^{siEV}PRA versus traditional bulk-analysis methods offers an alternative assay for analyzing biomarker heterogeneity in EVs. An advanced engineered EV model system was used to test the ability of the ^{siEV}PRA to measure differences in vesicular RNA. Three non-human miRNAs, including miR-39, miR-54, and miR-238 were engineered in EVs whereby their heterogeneity was detected. Similarly, the analysis of tetraspanin co-expression, which are abundant protein biomarkers in EVs, was analyzed on siEVs demonstrating low colocalization efficiencies, which agrees with reported siEV tetraspanin analyses. Although tetraspanins are highly expressed on EVs implying high co-expression, the siEV analysis demonstrated a heterogeneous expression of different tetraspanins not detectable with bulk characterization methods. Interestingly, when multiplexing across various biomolecule species, the colocalization efficiency was lower for protein-RNA detection compared to RNA-RNA and protein-protein detection. The difference may be attributed to the location of the biomolecules, since RNAs exist within the aqueous core of the EV, whereas tetraspanins are typically localized on the EV membrane surface. Regardless, the successful multiplexing of protein and RNA by the ^{siEV}PRA can expand the field of EV heterogeneity analyses.

With the ^{siEV}PRA, subpopulations of EVs from GBM cell lines demonstrated vesicular heterogeneity in protein, mRNA, and miRNA expression through colocalization analyses and were validated by bulk RNA sequencing. A comparative molecular analysis between RNA sequencing and the ^{siEV}PRA showed the possibility of integrating workflows for the discovery and validation of disease-specific RNA biomarkers, especially for RNA species enriched in EVs. Bulk RNA sequencing of GBM cell lines and their corresponding EVs revealed a subset of RNAs present at different concentrations among EVs and their parental cells. Some of the RNAs analyzed exhibited higher concentrations within EVs, which may be a result of selective packing or novel EV subtypes. This further emphasizes the importance of understanding the complex and diverse biogenesis of EVs and the mechanisms of molecular packing. Interestingly, for the miRNAs measured, a similar trend in the concentrations was observed between bulk sequencing and the SiEVPRA; however, some discrepancies were observed between microarrays and the ^{siEV}PRA for mRNA measurement. Given that mRNAs are long, single-stranded RNA molecules, whereas miRNAs are small single-stranded non-coding molecules, miRNA may hybridize more efficiently with the MBs as they have similar base-pair lengths. Perhaps generating MBs targeting different regions of the mRNA as was done for AXL would improve the consistency between bulk and siEV mRNA analyses.

The feasibility of analyzing RNAs in siEVs from GBM patient serum samples unveils the potential of the ^{siEV}PRA technology for various liquid biopsy applications with unmatched levels of sensitivity. Applying GBM patient serum to the ^{siEV}PRA, different mRNAs and miRNAs associated with GBM were validated and demonstrated the assay's clinical potential. Although the current study focused on cancer biomarker analysis, the ^{siEV}PRA can be easily adapted to other diseases. Furthermore, changing the capture antibodies within the microdomains can be used to sort EVs based on subpopulations, which may uncover differences in subpopulation-dependent packing of biomolecules and illuminate biogenesis pathways that conventional bulk-analysis methods may muddle. Lastly, the ability for the ^{siEV}PRA to multiplex across various biomolecule species offers a unique opportunity to study EV heterogeneity more comprehensively than has been previously accomplished.

1. A method of detecting a target type of molecular cargo with single extracellular vesicle resolution, the method comprising:
coating a glass substrate with polyethylene glycol (PEG) to create a PEG-coated glass substrate;
illuminating the PEG-coated glass substrate with UV light to create a micropattern array on the PEG-coated glass substrate;
attaching one or more capture antibodies to the micropattern array;
tethering a plurality of extracellular vesicles to the micropattern array via the one or more attached capture antibodies;
binding detection antibodies and molecular beacons to the tethered extracellular vesicles on the micropattern array, wherein the detection antibodies are configured to bind to a first target type of molecular cargo and the molecular beacons are configured to bind to a second target type of molecular cargo;
fluorescently imaging the micropattern array to capture image data; and
detecting occurrences of individual extracellular vesicles expressing the first target type of molecular cargo based on fluorescent spots of a first color associated with the detection antibodies in the captured image data;
detecting occurrences of individual extracellular vesicles expressing the second target type of molecular cargo based on fluorescent spots of a second color associated with the molecular beacons in the captured image data; and
detecting occurrences of individual extracellular vesicles expressing both the first target type of molecular cargo and the second target type of molecular cargo based on fluorescent spots of a third color in the captured image data.

2. The method of claim 1, wherein the glass substrate is coated with poly-L-lysine (PLL) through physisorption prior to coating the glass substrate with PEG, wherein the PEG is covalently bound to the PLL through N-hydroxysuccinimide (NHS) chemistry.

3. The method of claim 1, wherein the PEG is methoxy-poly(ethylene glycol)-succinimidyl valerate (mPEG-SVA).

4. The method of claim 1, wherein the micropattern array comprises an array of circles, wherein each individual circle has a diameter ranging from about 10 μm to about 100 μm.

5. The method of claim 4, wherein each individual circle has a diameter of about 20 μm.

6. The method of claim 1, wherein the micropattern array comprises an array of circles, wherein each individual circle has a center-to-center spacing of about 80 μm in relation to an adjacent circle.

7. The method of claim 1, wherein the one or more capture antibodies are biotinylated and attach to the micropattern array by binding to a physisorbed avidin layer on the micropattern array.

8. The method of claim 1, wherein the first color associated with the detection antibodies in the captured image data is in a first channel, wherein the second color associated with the molecular beacons in the captured image data is in a second channel, and wherein the third color in the captured image data is in a third channel.

9. The method of claim 1, wherein the first target type of molecular cargo includes a target type of protein, wherein the detection antibodies include detection antibodies configured to bind the target type of protein, and wherein the second target type of molecular cargo includes a target type of RNA, wherein the molecular beacons include molecular beacons configured to bind the target type of RNA.

10. The method of claim 9, wherein the target type of RNA is selected from microRNA (miRNA), messenger RNA (mRNA), other RNA types, or combinations thereof.

11. The method of claim 1, wherein the detection antibodies are conjugated with one or more fluorophores for fluorescent imaging.

12. The method of claim 1, wherein the molecular beacons comprise one or more fluorescent dye sequences for fluorescent imaging.

13. The method of claim 1, wherein the molecular beacons comprise one or more locked nucleic acid (LNA) nucleotides to improve thermal stability and nuclease resistance.

14. The method of claim 1, wherein the molecular beacons are selected from any one of SEQ ID NO: 1-13.

15. The method of claim 1, wherein fluorescently imaging the micropattern array to capture image data comprises total internal reflection fluorescence microscopy (TIRFM).

16. The method of claim 1, wherein the plurality of extracellular vesicles is present at a concentration of about 1.0×10⁶ extracellular vesicles/mL or greater.

17. A method of detecting a target type of molecular cargo with single extracellular vesicle resolution, the method comprising:
tethering a plurality of extracellular vesicles to a microarray;
binding a first plurality of detection probes and a second plurality of detection probes to the plurality of extracellular vesicles on the microarray, wherein the first plurality of detection probes are configured to bind to a first target type of molecular cargo and to appear as a first color in fluorescent imaging, and wherein the second plurality of detection probes are configured to bind to a second target type of molecular cargo and to appear as a second color in fluorescent imaging;
using fluorescent imaging to capture image data of the extracellular vesicles in the microarray at a resolution sufficient to differentiate individual extracellular vesicles;
detecting an occurrence of an extracellular vesicle expressing the first target type of molecular cargo by detecting a first color fluorescent spot in the captured image data;
detecting an occurrence of an extracellular vesicle expressing the second target type of molecular cargo by detecting a second color fluorescent spot in the captured image data; and
detecting an occurrence of an extracellular vesicle expressing both the first target type of molecular cargo and the second target type of molecular cargo by detecting a third color fluorescent spot in the captured image data.

18. A system for detecting a target type of molecular cargo with single extracellular vesicle resolution, the system comprising:
a polyethylene glycol (PEG)-coated glass substrate comprising a micropattern array having one or more capture antibodies attached thereto, wherein the one or more capture antibodies are configured to tether a plurality of extracellular vesicles;
a plurality of detection antibodies and a plurality of molecular beacons, wherein the plurality of detection antibodies and the plurality of molecular beacons are configured to bind the plurality of extracellular vesicles, wherein the plurality of detection antibodies are configured to bind to a first target type of molecular cargo, and wherein the plurality of molecular beacons are configured to bind to a second target type of molecular cargo; and
a fluorescent imaging device to capture image data;
wherein:
occurrences of individual extracellular vesicles expressing the first target type of molecular cargo are detected based on fluorescent spots of a first color associated with the detection antibodies in the captured image data;
occurrences of individual extracellular vesicles expressing the second target type of molecular cargo are detected based on fluorescent spots of a second color associated with the molecular beacons in the captured image data; and
occurrences of individual extracellular vesicles expressing both the first target type of molecular cargo and the second target type of molecular cargo are detected based on fluorescent spots of a third color in the captured image data.

19. The system of claim 18, wherein the PEG is methoxy-poly(ethylene glycol)-succinimidyl valerate (mPEG-SVA).

20. The system of claim 18, wherein the micropattern array comprises an array of circles, wherein each individual circle has a diameter ranging from about 10 μm to about 100 μm.

21-31. (canceled)


SEQUENCE LISTING (13 sequences)

SEQ ID NO: 1 - DNA, 36 bp, synthetic construct
  source 1..36: mol_type = other DNA
tcaacatcagtctgataagctaacttatcagactga

SEQ ID NO: 2 - DNA, 36 bp, synthetic construct; identical to SEQ ID NO: 1
  source 1..36: mol_type = other DNA

SEQ ID NO: 3 - DNA, 32 bp, synthetic construct
  source 1..32: mol_type = other DNA
ctccccggatttggcactgcagtgccaaatcc

SEQ ID NO: 4 - DNA, 33 bp, synthetic construct
  source 1..33: mol_type = other DNA
gtgattctgagctggctgaccaagccagctcag

SEQ ID NO: 5 - DNA, 36 bp, synthetic construct
  source 1..36: mol_type = other DNA
tggtgtctagttagtcacaactgtgtgactaactag

SEQ ID NO: 6 - DNA, 35 bp, synthetic construct
  source 1..35: mol_type = other DNA
catacagctagataaccaaagattggttatctagc

SEQ ID NO: 7 - DNA, 29 bp, synthetic construct
  source 1..29: mol_type = other DNA
gttgcccactgagaaggccttctcagtgg

SEQ ID NO: 8 - DNA, 31 bp, synthetic construct
  source 1..31: mol_type = other DNA
gctccaggcatatccacctcatggatatgcc

SEQ ID NO: 9 - DNA, 31 bp, synthetic construct
  source 1..31: mol_type = other DNA
cctgctccaaaaatccattcggatttttgga

SEQ ID NO: 10 - DNA, 32 bp, synthetic construct
  source 1..32: mol_type = other DNA
caagcgatttacacccggtgagggtgtaaatc

SEQ ID NO: 11 - DNA, 35 bp, synthetic construct
  source 1..35: mol_type = other DNA
gttctcgtcgtctcatatcctggatatgagacgac

SEQ ID NO: 12 - DNA, 33 bp, synthetic construct
  source 1..33: mol_type = other DNA
ctttgaacgtccgagaacatccattctcggacg

SEQ ID NO: 13 - DNA, 32 bp, synthetic construct
  source 1..32: mol_type = other DNA
ctccgtcatgtgctgtgacttcacagcacatg